(19) United States
(12) Patent Application Publication
(10) Pub. No.: US 2024/0252621 A1
(43) Pub. Date: Aug. 1, 2024

(54) VIRUS-LIKE PARTICLE VACCINE FOR CORONAVIRUS

(71) Applicant: Icosavax, Inc., Seattle, WA (US)

(72) Inventors: Niranjan Kanesa-Thasan, Seattle, WA (US); Charles Richardson, Seattle, WA (US)

(21) Appl. No.: 18/565,728

(22) PCT Filed: Jun. 3, 2022
(86) PCT No.: PCT/US2022/032201

§ 371 Date: Nov. 30, 2023

Related U.S. Application Data
(60) Provisional application No. 63/197,952, filed on Jun. 7, 2021.

(51) Int. Cl. A61K 39/215; A61K 39/00; A61K 39/39; A61P 37/04

(52) U.S. Cl. CPC A61K 39/215; A61K 39/39; A61P 37/04; A61K 2039/545; A61K 2039/55505; A61K 2039/55561; A61K 2039/55566; A61K 2039/6031

(57) ABSTRACT

The present disclosure relates to targeting SARS-CoV-2, in particular, prevalent strains of SARS-CoV-2, and methods of using such vaccines to induce neutralizing antibody levels against SARS-CoV-2.

### RELATED APPLICATIONS

This application claims the benefit of priority under 35 U.S.C. § 119(e) to U.S. Provisional Application No. 63/197,952 filed Jun. 7, 2021 the entire contents of which is incorporated herein by reference in its entirety.

### FIELD OF THE DISCLOSURE

The present disclosure relates to targeting SARS-CoV-2, in particular, prevalent strains of SARS-CoV-2, and methods of using such vaccines to induce neutralizing antibody levels against SARS-CoV-2.

### INCORPORATION BY REFERENCE OF SEQUENCE LISTING

This application contains a Sequence Listing which has been submitted in ASCII format via EFS-WEB and is hereby incorporated by reference in its entirety. Said ASCII copy, created on Jun. 2, 2022 is named 061291-505001WO_ST25.txt and is 64 kilobytes in size.

### BACKGROUND

Severe acute respiratory syndrome coronavirus 2 (SARS-CoV-2) is a viral pathogen responsible for the coronavirus disease 2019 (COVID-19) global pandemic. As of May 2022, there were over 500 million cumulative cases and over 6.2 million deaths from COVID-19 worldwide with over 1 million deaths in the United States alone. Rates of serious morbidity and mortality from COVID-19 are disproportionately higher in older adults as compared to other age groups, likely due to age-induced immunosenescence. Despite the fact that adults over 65 constitute 17% of the United States population, over 75% of the deaths in the United States due to COVID-19 have been in this age group.

Vaccines have been developed to combat this pandemic at an unprecedented pace and there are several SARS-CoV-2 vaccines that have been licensed or approved under emergency use authorization. The initial push for first wave vaccines to fight the pandemic has focused on speed rather than other critical attributes that are now important considerations for second wave vaccine candidates such as durability, potential to boost response, potential to address variant strains, ease of manufacturing and distribution, stability, and reactogenicity profile.

Coronaviruses are prone to mutation but the pace at which the SARS-CoV-2 virus has mutated is faster than most were anticipating. Some of these emerging strains appear to enhance transmission and pathogenicity, with complete replacement of the original SARS-CoV-2 strain by the emerging strains in some countries. Data has shown that some vaccines against the original SARS-CoV-2 virus strain are less immunogenic against some of the emerging variants, particularly the B.1.351 (beta) and B.1.1.529 (omicron) variants first identified in South Africa. Decreases in neutralizing titers against the B.1.351 and B.1.1.529 strains in vitro appear to translate to lower efficacy in people who are infected with these virus strains. Others initiated efforts to supplement existing vaccines to address emerging variants with either booster shots or new vaccines incorporating key mutations found in variant strains. However, initial exposure to the original strain through natural infection or vaccination may result in a focusing of the immune system on the original strain in such a way as to interfere with the development of an immune response against the new strain, a phenomenon called “original antigenic sin”.

Virus-like particle (VLP) vaccines allow for stable multivalent antigen display, facilitating cross-linking of B-cell receptors and driving stronger immune signaling than soluble protein antigens. VLP vaccines have historically been shown to induce durable immunity [e.g. human papilloma virus (HPV)] and there are several examples of licensed vaccines utilizing naturally-occurring self-assembling VLPs, including human papilloma virus (HPV) and hepatitis B (HBV) vaccines.

There is a need for novel vaccines targeting the prevalent and emerging SARS-CoV-2 strains to induce high neutralizing antibody levels. The compositions and methods of the present disclosure address that need.

### BRIEF SUMMARY

In one aspect, provided herein is a pharmaceutical composition, comprising a protein complex comprising a first component comprising a receptor-binding domain of a coronavirus S protein and a first multimerization domain, and optionally a second component comprising a second multimerization domain; and one or more pharmaceutically acceptable diluents or excipients.

In some embodiments, the pharmaceutical composition comprises an adjuvant. In some embodiments, the adjuvant is a squalene-in-water emulsion. In some embodiments, the adjuvant is MF59®. In some embodiments, the adjuvant comprises an oil-in-water emulsion.

In some embodiments, the protein complex is an icosahedral protein complex. In some embodiments, the first multimerization domain comprises an amino acid sequence which is at least 75%, 80%, 85%, 90%, 91%, 92%, 93%, 94%, 95%, 96%, 97%, 98%, 99%, or 100% identical to an amino acid sequence selected from the group consisting of SEQ ID NOS: 9-13 or 18. In some embodiments, the second multimerization domain comprises an amino acid sequence which is at least 95%, at least 96%, at least 97%, at least 98%, at least 99%, or at least 100% identical to an amino acid sequence selected from the group consisting of SEQ ID NOS: 14-17, 20 or 27. In some embodiments, the first component comprises an amino acid sequence which is at least 75%, 80%, 85%, 90%, 91%, 92%, 93%, 94%, 95%, 96%, 97%, 98%, 99%, or 100% identical to any one of SEQ ID NOs: 1-6; and the second component comprises an amino acid sequence which is at least 75%, 80%, 85%, 90%, 91%, 92%, 93%, 94%, 95%, 96%, 97%, 98%, 99%, or 100% identical to SEQ ID NO: 14.

In another aspect, provided herein is a unit dose of the pharmaceutical composition described herein, wherein the unit dose comprises 2 μg, 5 μg, 10 μg, 15 μg, 25 μg, 50 μg, 100 μg, or 125 μg of the protein complex. In some embodiments, provided herein is a unit dose of the pharmaceutical composition described herein, wherein the unit dose comprises between about 25 μg and about 125 μg of the protein complex. In some embodiments, the unit dose of the pharmaceutical composition is between about 2 μg to about 125 μg, or between about 5 μg to about 125 g, or between about 15 μg to 125 μg, or between about 25 μg to about 125 μg, or between about 50 μg to about 125 μg, or between about 100 μg to about 125 μg of the protein complex.

In some embodiments, the disclosure provides a pharmaceutical composition, comprising a protein complex comprising a first component comprising a receptor-binding domain of a coronavirus S protein attached to a first multimerization domain, and optionally a second component comprising a second multimerization domain; and optionally one or more pharmaceutically acceptable diluents or excipients.

In some embodiments, the pharmaceutical composition comprises an adjuvant.

In some embodiments, the adjuvant is a squalene-in-water emulsion.

In some embodiments, the adjuvant is MF59®.

In some embodiments, the adjuvant is an aluminum salt.

In some embodiments, the adjuvant is CPG-1018.

In some embodiments, the pharmaceutical composition comprises both an aluminum salt and CPG-1018.

In some embodiments, the pharmaceutical composition is free of or substantially free of any adjuvant.

In some embodiments, the first multimerization domain is a trimerization domain and the second multimerization domain is a pentamerization domain.

In some embodiments, the protein complex is an icosahedral protein complex.

In some embodiments, the first multimerization domain comprises an amino acid sequence which is at least 75%, 80%, 85%, 90%, 91%, 92%, 93%, 94%, 95%, 96%, 97%, 98%, 99%, or 100% identical to an amino acid sequence selected from the group consisting of SEQ ID NOS: 9-13 or 18.

In some embodiments, the second multimerization domain comprises an amino acid sequence which is at least 95%, at least 96%, at least 97%, at least 98%, at least 99%, or at least 100% identical to an amino acid sequence selected from the group consisting of SEQ ID NOS: 14-17, 20 or 27.

In some embodiments, the first component comprises an amino acid sequence which is at least 75%, 80%, 85%, 90%, 91%, 92%, 93%, 94%, 95%, 96%, 97%, 98%, 99%, or 100% identical to any one of SEQ ID NOs: 1-6; and wherein the second component comprises an amino acid sequence which is at least 75%, 80%, 85%, 90%, 91%, 92%, 93%, 94%, 95%, 96%, 97%, 98%, 99%, or 100% identical to SEQ ID NO: 14.

In some embodiments, the disclosure provides a unit dose of the pharmaceutical composition of any one of embodiments 1 to 13, wherein the unit dose comprises 2 μg, 5 μg, 10 μg, 15 μg, 25 μg, 50 μg, 100 μg, or 125 μg of the protein complex.

In some embodiments, the disclosure provides a method of vaccinating a subject at risk of infection with SARS-CoV-2, comprising administering to the subject a pharmaceutical composition comprising an effective amount of a protein complex comprising a first component comprising a receptor-binding domain of a coronavirus S protein attached to a first multimerization domain, and a second component comprising a second multimerization domain; and one or more pharmaceutically acceptable diluents or excipients.

In some embodiments, the disclosure provides a method of boosting an immune response to a prior vaccination for SARS-CoV-2, comprising administering to a subject previously vaccinated for SARS-CoV-2 a pharmaceutical composition comprising an effective amount of a protein complex comprising a first component comprising a receptor-binding domain of a coronavirus S protein attached to a first multimerization domain, and optionally a second component comprising a second multimerization domain.

In some embodiments, the subject has been previously vaccinated with a full vaccination course of a primary vaccine.

In some embodiments, the disclosure provides a method of safely and effectively immunizing a subject for SARS-CoV-2, comprising administering to a subject previously vaccinated for SARS-CoV-2 a pharmaceutical composition comprising an effective amount of a protein complex comprising a first component comprising a receptor-binding domain of a coronavirus S protein attached to a first multimerization domain, and optionally a second component comprising a second multimerization domain.

In some embodiments, the pharmaceutical composition comprises an adjuvant.

In some embodiments, the adjuvant is a squalene-in-water emulsion.

In some embodiments, the adjuvant is MF59®.

In some embodiments, the adjuvant is an aluminum salt.

In some embodiments, the adjuvant is CPG-1018.

In some embodiments, the pharmaceutical composition comprises both an aluminum salt and CPG-1018.

In some embodiments, the pharmaceutical composition is free of or substantially free of any adjuvant.

In some embodiments, the first multimerization domain is a trimerization domain and the second multimerization domain is a pentamerization domain.

In some embodiments, the protein complex is an icosahedral protein complex.

In some embodiments, the first multimerization domain comprises an amino acid sequence which is at least 75%, 80%, 85%, 90%, 91%, 92%, 93%, 94%, 95%, 96%, 97%, 98%, 99%, or 100% identical to an amino acid sequence selected from the group consisting of SEQ ID NOS: 9-13 or 18.

In some embodiments, the second multimerization domain comprises an amino acid sequence which is at least 95%, at least 96%, at least 97%, at least 98%, at least 99%, or at least 100% identical to an amino acid sequence selected from the group consisting of SEQ ID NOS: 14-17, 20 or 27.

In some embodiments, the first component comprises an amino acid sequence which is at least 75%, 80%, 85%, 90%, 91%, 92%, 93%, 94%, 95%, 96%, 97%, 98%, 99%, or 100% identical to any one of SEQ ID NOs: 1-6; and wherein the second component comprises an amino acid sequence which is at least 75%, 80%, 85%, 90%, 91%, 92%, 93%, 94%, 95%, 96%, 97%, 98%, 99%, or 100% identical to SEQ ID NO: 14.

In some embodiments, the effective amount is 2 μg, 5 μg, 10 μg, 15 μg, 25 μg, 50 μg, 100 μg, or 125 μg of the protein complex.

In some embodiments, the method comprises repeating the administering step.

In some embodiments, the method comprises administering a booster vaccine.

In some embodiments, the method comprises administering a prime vaccine.

In some embodiments, the prime vaccine is an mRNA-based vaccine, an adenoviral vector-based vaccine, a protein-based vaccine, or an inactivated virus vaccine.

In some embodiments, the prime vaccine is the protein complex.

In some embodiments, the subject is a previously vaccinated subject.

In some embodiments, the subject has completed a full course of vaccination for an original strain of SARS-CoV-2.

In some embodiments, the subject has completed a partial course (e.g., has received one of two doses) of vaccination for an original strain of SARS-CoV-2.

In some embodiments, the subject has received at least one dose of a vaccination for a variant strain of SARS-CoV-2.

In some embodiments, the subject has received at least one dose of a vaccine comprising the receptor binding domain of a coronavirus S protein or a polynucleotide encoding the receptor binding domain of a coronavirus S protein.

In some embodiments, the subject has received at least one dose of a vaccine comprising a coronavirus S protein or a polynucleotide encoding a coronavirus S protein.

In some embodiments, the coronavirus S protein is S2P.

In some embodiments, the S protein is HexaPro.

In some embodiments, the subject is a vaccination naïve subject.

In some embodiments, the subject has previously been infected with SARS-CoV-2.

In some embodiments, the subject has not previously been infected with SARS-CoV-2.

In some embodiments, the subject does not have antibodies against SARS-CoV-2 prior to the administering step.

In some embodiments, the subject has antibodies against SARS-CoV-2 prior to the administering step.

In some embodiments, the method induces neutralizing antibody titers in the subject.

In some embodiments, the method induces S protein-specific and IgG antibody titers in the subject.

In some embodiments, the method prevents infection with SARS-CoV-2.

In some embodiments, the method prevents infection with an original strain of SARS-CoV-2.

In some embodiments, the method prevents infection with a variant strain of SARS-CoV-2.

In some embodiments, the method reduces the severity of infection with coronavirus.

In some embodiments, the method reduces the severity of infection with an original strain of SARS-CoV-2.

In some embodiments, the method reduces the severity of infection with a variant strain of SARS-CoV-2.

### BRIEF DESCRIPTION OF THE SEVERAL VIEWS OF THE DRAWINGS

FIG. 1 shows a structural model of assembly of a vaccine from a first component including an antigenic fragment (here: the receptor binding domain) of the S protein (CompA-RBD-01) and a second component (CompB).

FIG. 2 shows a summary of a clinical trial design.

FIG. 3 is a schematic showing an IVX-411 Phase 1/2 trial study overview. The topline data includes two components.

FIG. 4 is a schematic showing an IVX-411 Phase 1/2 study design.

FIGS. 5A and 5B are graphs showing local (FIG. 5A) and systemic (FIG. 5B) adverse events (AEs) within 7 days of any dose in Parts 1 and 2 of the study.

FIGS. 6A and 6B are graphs showing neutralizing and spike IgG antibody titers in Part 1
SARS-CoV-2-naïve subjects (FIG. 6A) and Part 2—previously vaccinated subjects (FIG. 6B) of the study.

FIGS. 7A and 7B are graphs showing wild type and omicron neutralizing antibody titers in Part 1—SARS-CoV-2 naive subjects (FIG. 7A) and Part 2—previously vaccinated subjects (FIG. 7B) of the study.

### DETAILED DESCRIPTION

Provided herein are pharmaceutical compositions comprising a protein complex comprising which may be used in the treatment of SARS-CoV2.

The term “a” or “an” refers to one or more of that entity, i.e. can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein the term “sequence identity” refers to the extent to which two optimally aligned polynucleotides or polypeptide sequences are invariant throughout a window of alignment of residues, e.g. nucleotides or amino acids. An “identity fraction” for aligned segments of a test sequence and a reference sequence is the number of identical residues which are shared by the two aligned sequences divided by the total number of residues in the reference sequence segment, i.e. the entire reference sequence or a smaller defined part of the reference sequence. “Percent identity” is the identity fraction times 100. Comparison of sequences to determine percent identity can be accomplished by a number of well-known methods, including for example by using mathematical algorithms, such as, for example, those in the BLAST suite of sequence analysis programs. Unless noted otherwise, the term “sequence identity” refers to sequence identity as calculated by Blast-p program of the National Center for Biotechnology Information (NCBI) online alignment tool, version 2.11.0 (released Oct. 19, 2020). Altschul et al. J. Mol. Biol. 215:403-410 (1990).

As used herein, the terms “heterologous vaccine” and “heterologous vaccination” refer to a vaccine given to a subject who has received or will receive a vaccination for the same indication (e.g., COVID19) using a vaccine made with another technology (e.g., an mRNA vaccine, adenoviral vector vaccine, or a protein based vaccine). As such, a “heterologous vaccine” refers to a vaccine made using a different technology type than the reference vaccine.

A “heterologous boost” or “heterologous boost vaccine” refers to a heterologous vaccine (e.g., a protein-based VLPs) given to a subject who has received a vaccination for the same indication (e.g., COVID19) using a vaccine made with another technology (e.g., an mRNA vaccine, adenoviral vector vaccine, or a protein based vaccine).

The term “prime vaccine” refers to the first vaccine in a vaccination protocol or to a first set of vaccines administered prior to a heterologous boost vaccine. For example an mRNA vaccine or adenoviral vaccine may be administered first, optionally followed by a second prime vaccine after a suitable interval, and then the heterologous vaccine may be administered. The heterologous vaccine may serve to “boost” the immune response to the prime vaccine. A “priming vaccine” as used herein refers to a vaccine comprising an agent(s) that encodes the target antigen to which an immune response is to be generated. Priming vaccines are administered to the subject in an amount effective to elicit an immune response to the target antigen.

A “heterologous prime-boost vaccination” refers to a vaccine given to a subject who will receive a vaccination for the same indication (e.g., COVID19) using a vaccine made with another technology. For example, the initial dose (primary vaccine or prime vaccination) of a vaccine may an mRNA vaccine (or alternatively, the subject may have been diagnosed with the indication e.g., COVID19), and subsequently receive a second vaccination for the same indication, wherein the second vaccination is of a different technology—a heterologous vaccination (e.g., a protein-based VLP). In examples, heterologous prime-boost vaccination includes a primary vaccination for an indication, and a subsequent vaccination for the same indication, wherein the heterologous vaccination is administered 3 months to 6 months after the heterologous prime vaccine, or 4 or more months after a heterologous prime vaccine, or 6 months or more after a heterologous prime vaccine, or 10 months or more after a heterologous prime vaccine. In yet other examples, the heterologous boost vaccination is administered 1 year after a heterologous prime vaccine. A “heterologous prime” or “heterologous prime vaccine” refers to a vaccine given to a subject who will receive a vaccination for the same indication (e.g., COVID19) using a vaccine made with another technology (e.g., an mRNA vaccine, adenoviral vector vaccine, or a protein subunit vaccine).

The term “HexaPro” refers to a S protein four beneficial proline substitutions (F817P, A892P, A899P, A942P) as well as the two proline substitutions in S-2P (prolines at positions 986 and 987). See Hsich et al. Science 369:1501-05 (2020). In some embodiments, the subject is a vaccination naive or SARS-CoV-2 uninfected subject. In some embodiments, the vaccine is an mRNA-based vaccine, an adenoviral vector-based vaccine, a protein-subunit based vaccine, or an inactivated virus vaccine. As used herein, a “subunit” composition, for example a vaccine, that includes one or more selected antigens but not all antigens from a pathogen.

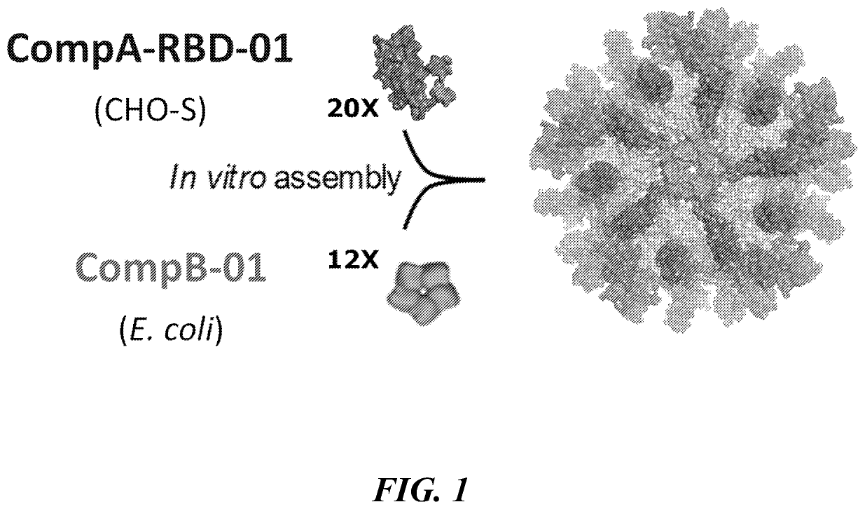

The term “virus-like particle” or “VLP” refers to a molecular assembly that resembles a virus, but is non-infectious, and that displays an antigenic protein, or antigenic fragment thereof, of a viral protein or glycoprotein. A “protein-based VLP” refers to a VLP formed from proteins or glycoproteins and substantially free of other components (e.g., lipids). Protein-based VLPs may include post-translation modification and chemical modification, but are to be distinguished from micellar VLPs and VLPs formed by extraction of viral proteins from live or live inactivated virus preparations. The term “designed VLP” refers to a VLP comprising one or more polypeptides generated by computational protein design. Illustrative designed VLP are VLPs that comprise nanostructures depicted in FIG. 1. The term “symmetric VLP” refers to a protein-based VLP with a symmetric core, such as shown in FIG. 1. These include but are not limited to designed VLPs. For example, the protein ferritin has been used to generate a symmetric, protein-based VLP using naturally occurring ferritin sequences. Ferritin-based VLPs are distinguished from designed VLPs in that no protein engineering is necessary to form a symmetric VLP from ferritin, other than fusing the viral protein to the ferritin molecule. Protein design methods can be used to generate similar one- and two-component nanostructures based on template structures (e.g., structures deposited in the Protein Data Bank) or de novo (i.e., by computational design of new proteins having a desired structure but little or no homology to naturally occurring proteins). Such one- and two-component nanostructures can then be used as the core of a designed VLP. The terms “protein nanoparticle” or “nanoparticle” and the term “nanostructure” may be used to refer to protein-based VLPs as described herein.

As used herein, an “immunogenic composition” is a composition that comprises an antigen where administration of the composition to a subject results in the development in the subject of a humoral and/or a cellular immune response to the antigen.

As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular aspects, the subject is up to 4 months old, or up to 6 months old. In some aspects, the adults are seniors about 65 years or older, or about 60 years or older. In some aspects, the subject is a pregnant woman or a woman intending to become pregnant. In other aspects, subject is not a human; for example a non-human primate; for example, a baboon, a chimpanzee, a gorilla, or a macaque. In certain aspects, the subject may be a pet, such as a dog or cat.

The present disclosure relates generally to vaccination of a subject with a protein complex (e.g., a protein-based Virus-like Particle) comprising a first component comprising a receptor-binding domain of a coronavirus spike (S) protein, or alternatively another antigenic portion of the coronavirus S protein, and a first multimerization domain.

In December 2019, a pneumonia outbreak of unknown cause occurred in Wuhan, China and it became clear that a novel coronavirus (severe acute respiratory syndrome coronavirus 2; SARS-CoV-2) was the underlying cause. The genetic sequence of SARS-CoV-2 became available to the WHO and public (MN908947.3) and the virus was categorized into the betacoronavirus subfamily. By sequence analysis, the phylogenetic tree revealed a closer relationship to severe acute respiratory syndrome (SARS) virus isolates than to other coronaviruses that infect humans, such as the Middle East respiratory syndrome (MERS) virus.

Coronaviruses are positive-sense, single-stranded RNA ((+)ssRNA) enveloped viruses that encode for a total of four structural proteins, spike protein (S), envelope protein (E), membrane protein (M) and nucleocapsid protein (INI). The spike protein (S protein) is responsible for receptor-recognition, attachment to the cell, infection via the endosomal pathway, and the genomic release driven by fusion of viral and endosomal membranes. Though sequences between the different family members vary, there are conserved regions and motifs within the S protein making it possible to divide the S protein into two subdomains: S1 and S2. While the S2, with its transmembrane domain, is responsible for membrane fusion, the S1 domain recognizes the virus-specific receptor and binds to the target host cell. The structure of the SARS-CoV-2 S protein, including its receptor binding domain (RBD), has been determined by cyro-electron microscopy (Cyro-EM) (Wrapp et al. Science 367:1260-1263 (2020)).

The S protein portion and the first multimerization domain may be linked by any suitable means, including co-expression as a fusion protein. The protein complex may optionally comprise a second component comprising a second multimerization domain. The pharmaceutical composition typically comprises one or more pharmaceutically acceptable diluents or excipients. The antigenic portion of the first component may comprise, consist essentially of, or consist of a selected fragment of the coronavirus S protein. For example, the antigenic portion may comprise the receptor binding domain of the coronavirus S protein with flanking sequences on the domain's N or C terminus (e.g., 5, 10, 20, 30, or more amino acids of the coronavirus S protein outside the receptor binding domain); or the antigenic portion may include only a few flanking amino acids (e.g., 1, 2, 3, 4, or 5 amino acids from the coronavirus S protein); or the antigenic portion may include only the receptor binding domain with no flanking sequences from the coronavirus S protein.

In some embodiments, the protein complex is an icosahedral protein complex, such as those disclosed in U.S. Pat. No. 10,248,758 or U.S. Patent Pub. No. 2020/0392187 A1, the contents of which are incorporated by reference herein in their entireties.

The multimerization domains may be derived from a naturally-occurring protein sequence by substitution of at least one amino acid residue or by additional at the N- or C-terminus of one or more residues. In some cases, the first multimerization domain comprises a protein sequence determined by computational methods. This first multimerization domain may form the entire core of the VLP; or the core of the VLP may comprise one or more additional polypeptides (also referred to a “second component” or third, fourth, fifth component and so on), such that the VLP comprises two, three, four, five, six, seven, or more multimerization domains. In some cases, the first component will form trimers related by 3-fold rotational symmetry and the second component will form pentamers related by 5-fold rotational symmetry. In such cases, the VLP forms an “icosahedral particle” having 153 symmetry. Together these one or more pluralities of component may be arranged such that the members of each plurality of component are related to one another by symmetry operators. A general computational method for designing self-assembling protein materials, involving symmetrical docking of protein building blocks in a target symmetric architecture, is disclosed in U.S. Patent Pub. No. US 2015/0356240 A1.

The “core” of the VLP is used herein to describe the central portion of the VLP that links together the several copies of the RBD or coronavirus S protein ectodomain, or antigenic fragments thereof, displayed by the VLP. In an embodiment, the first component comprises a first polypeptide comprising an RBD, a linker, and a first polypeptide comprising a multimerization domain.

In some cases, the VLP is adapted to display the RBD or S protein from two or more diverse strains of coronavirus. In non-limiting examples, the same VLP displays mixed populations of protein antigens or mixed heterotrimers of protein antigens from different strains of coronavirus.

The VLPs of the present disclosure display antigenic proteins in various ways including as gene fusion or by other means disclosed herein. As used herein, “linked to” or “attached to” denotes any means known in the art for causing two polypeptides to associate. The association may be direct or indirect, reversible or irreversible, weak or strong, covalent or non-covalent, and selective or nonselective.

In some embodiments, attachment is achieved by genetic engineering to create an N- or C-terminus fusion of an antigen to one of the pluralities of polypeptides composing the VLP. Thus, the VLP may consist of, or consist essentially of, one, two, three, four, five, six, seven, eight, nine, or ten pluralities of polypeptides displaying one, two, three, four, five, six, seven, eight, nine, or ten pluralities of antigens, where at least one of the pluralities of antigen is genetically fused to at least one of the plurality of polypeptides. In some cases, the VLP consists essentially of one plurality of polypeptides capable of self-assembly and comprising the plurality of antigenic proteins genetically fused thereto. In some cases, the VLP consists essentially of a first plurality of polypeptides comprising a plurality of antigens; and a second plurality of polypeptides capable of co-assembling into two-component VLP, one plurality of polypeptides linking the antigenic protein to the VLP and the other plurality of polypeptides promoting self-assembly of the VLP.

In some embodiments, attachment is achieved by post-translational covalent attachment between one or more pluralities of polypeptides and one or more pluralities of antigenic protein. In some cases, chemical cross-linking is used to non-specifically attach the antigen to a VLP polypeptide. In some cases, chemical cross-linking is used to specifically attach the antigenic protein to a VLP polypeptide (e.g. to the first polypeptide or the second polypeptide). Various specific and non-specific cross-linking chemistries are known in the art, such as Click chemistry and other methods. In general, any cross-linking chemistry used to link two proteins may be adapted for use in the presently disclosed VLPs. In particular, chemistries used in creation of immunoconjugates or antibody drug conjugates may be used. In some cases, an VLP is created using a cleavable or non-cleavable linker. Processes and methods for conjugation of antigens to carriers are provided by, e.g., U.S. Patent Pub. No. US 2008/0145373 A1.

The components of the VLP of the present disclosure may have any of various amino acids sequences. U.S. Patent Pub No. US 2015/0356240 A1 describes various methods for designing protein assemblies. As described in US Patent Pub No. US 2016/0122392 A1 and in International Patent Pub. No. WO 2014/124301 A1, the polypeptides were designed for their ability to self-assemble in pairs to form VLPs, such as icosahedral particles. The design involved design of suitable interface residues for each member of the polypeptide pair that can be assembled to form the VLP. The VLPs so formed include symmetrically repeated, non-natural, non-covalent polypeptide-polypeptide interfaces that orient a first assembly and a second assembly into a VLP, such as one with an icosahedral symmetry.

In some embodiments, the protein complex is a designed protein-based VLP as depicted in FIG. 1. The protein-based VLP may comprise the proteins described in Table 3 or functional variants thereof. The VLP may display the receptor-binding domain of a coronavirus spike (S) protein, such as SARS-CoV-2, or it may display an ectodomain of a coronavirus S protein. While certain representative protein-based VLPs are described herein, in variations, other protein-based VLPs may be used. The VLP may be a ferritin-based VLP. In some embodiments, the protein complex is a protein-based VLP (including ferritin, E2p, 13-01 and 13-01 variants) as described in U.S. Pat. Pub. No. US 2020/0009244 A1 and Int'l Pat. Pub. Nos. WO 2022/046583 A1 and WO 2021/210984 A1, the disclosures of which are incorporated by reference herein. The protein-based VLP may employ a variety of coupling techniques to attach an antigen to the VLP core, including but not limited to the SpyCatcher system described in, e.g., Escolano et al. Nature 570:468-473 (2019), He et al. Sci Adv. 7(12):cabf1591 (2021), and Tan et al. Nat. Commun. 12(1):542 (2021). The protein-based VLP may be a lumazine synthase nanoparticle as described, e.g., in Geng et al. PLOS Pathog. 17(9):e1009897 (2021). The protein-based VLP may be a ferritin nanoparticle as described, e.g., in Joyce et al. bioRxiv 2021.05.09.443331 and in U.S. Pat. Pub. No. US 2019/0330279 A1.

In some embodiments, the RBD or coronavirus S protein ectodomain, or antigenic fragments thereof, are expressed as a fusion protein with the first multimerization domain. In some embodiments, the first multimerization domain and RBD or coronavirus S protein ectodomain are joined by a linker sequence. In some embodiments, the linker sequence comprises a foldon, wherein the foldon sequence is EKAAKAEEAARK (SEQ ID NO: 8). In some embodiments, the linker may comprise a Gly-Ser linker (i.e. a linker consisting of glycine and serine residues) of any suitable length. In some embodiments, the Gly-Ser linker may be 3, 4, 5, 6, 7, 8, 9, 10, 11, 12, 13, 14, 15, 16, 17, 18, 19, 20, or more amino acids in length.

Non-limiting examples of designed protein complexes useful in protein-based VLPs of the present disclosure include those disclosed in U.S. Pat. No. 9,630,994; Int'l Pat. Pub No. WO2018187325A1; U.S. Pat. Pub. No. 2018/0137234 A1; U.S. Pat. Pub. No. 2019/0155988 A2, each of which is incorporated herein in its entirety. Illustrative sequences are provided in Table 3.

TABLE 3

| | | | Identified |
|---|---|---|---|
| | Component | | interface |
| Name | Multimer | Amino Acid Sequence | residues |
| I53-50A | trimer | EELFKKHKIVAVLRANSVEEAIEKAVA | I53-50A: |
| SEQ ID NO: 9 | | VFAGGVHLIEITFTVPDADTVIKALSVL | 25, 29, 33, 54, |
| | | KEKGAIIGAGTVTSVEQCRKAVESGAE | 57 |
| | | FIVSPHLDEEISQFCKEKGVFYMPGVMT | |
| | | PTELVKAMKLGHTILKLFPGEVVGPQF | |
| | | VKAMKGPFPNVKFVPTGGVNLDNVCE | |
| | | WFKAGVLAVGVGSALVKGTPDEVREK | |
| | | AKAFVEKIRGCTE | |
| I53-50A-Δcys | trimer | EELFKKHKIVAVLRANSVEEAIEKAVA | I53-50A: |
| SEQ ID NO: 28 | | VFAGGVHLIEITFTVPDADTVIKALSVL | 25, 29, 33, 54, |
| | | KEKGAIIGAGTVTSVEQARKAVESGAE | 57 |
| | | FIVSPHLDEEISQFAKEKGVFYMPGVMT | |
| | | PTELVKAMKLGHTILKLFPGEVVGPQF | |
| | | VKAMKGPFPNVKFVPTGGVNLDNVAE | |
| | | WFKAGVLAVGVGSALVKGTPDEVREK | |
| | | AKAFVEKIRGATELE | |
| I53-50A.1 | trimer | EELFKKHKIVAVLRANSVEEAIEKAVA | I53-50A: |
| SEQ ID NO: 10 | | VFAGGVHLIEITFTVPDADTVIKALSVL | 25, 29, 33, 54, |
| | | KEKGAIIGAGTVTSVEQCRKAVESGAE | 57 |
| | | FIVSPHLDEEISQFCKEKGVFYMPGVMT | |
| | | PTELVKAMKLGHDILKLFPGEVVGPQF | |
| | | VKAMKGPFPNVKFVPTGGVNLDNVCE | |
| | | WFKAGVLAVGVGDALVKGDPDEVRE | |
| | | KAKKFVEKIRGCTE | |
| I53- | trimer | EELFKKHKIVAVLRANSVEEAIEKAVA | I53-50A: |
| 50A.1NegT2 | | VFAGGVHLIEITFTVPDADTVIKALSVL | 25, 29, 33, 54, |
| SEQ ID NO: 11 | | KEKGAIIGAGTVTSVEQCRKAVESGAE | 57 |
| | | FIVSPHLDEEISQFCKEKGVFYMPGVMT | |
| | | PTELVKAMKLGHDILKLFPGEVVGPEF | |
| | | VEAMKGPFPNVKFVPTGGVDLDDVCE | |
| | | WFDAGVLAVGVGDALVEGDPDEVRED | |
| | | AKEFVEEIRGCTE | |
| I53-50A.1PosT1 | trimer | EELFKKHKIVAVLRANSVEEAIEKAVA | I53-50A: |
| SEQ ID NO: 12 | | VFAGGVHLIEITFTVPDADTVIKALSVL | 25, 29, 33, 54, |
| | | KEKGAIIGAGTVTSVEQCRKAVESGAE | 57 |
| | | FIVSPHLDEEISQFCKEKGVFYMPGVMT | |
| | | PTELVKAMKLGHDILKLFPGEVVGPQF | |
| | | VKAMKGPFPNVKFVPTGGVNLDNVCK | |
| | | WFKAGVLAVGVGKALVKGKPDEVRE | |
| | | KAKKFVKKIRGCTE | |
| I53-50A genus | trimer | EELFKKHKIVAVLRANSVEEAIEKAVA | |
| SEQ ID NO: 13 | | VFAGGVHLIEITFTVPDADTVIKALSVL | |
| | | KEKGAIIGAGTVTSVEQCRKAVESGAE | |
| | | FIVSPHLDEEISQFCKEKGVFYMPGVMT | |
| | | PTELVKAMKLGH(T/D)ILKLFPGEVVGP | |
| | | (Q/E)FV(K/E)AMKGPFPNVKFVPTGGV | |
| | | (N/D)LD(N/D)VC(E/K)WF(K/D)AGVLAV | |
| | | GVG(S/K/D)ALV(K/E)G(T/D/K)PDEVRE | |
| | | (K/D)AK(A/E/K)FV(E/K)(K/E)IRGCTE | |
| I53-50B | pentamer | NQHSHKDYETVRIAVVRARWHAEIVD | I53-50B: |
| SEQ ID NO: 14 | | ACVSAFEAAMADIGGDRFAVDVFDVP | 24, 28, 36, 124, |
| | | GAYEIPLHARTLAETGRYGAVLGTAFV | 125, 127, 128, |
| | | VNGGIYRHEFVASAVIDGMMNVQLST | 129, 131, 132, |
| | | GVPVLSAVLTPHRYRDSDAHTLLFLAL | 133, 135, 139 |
| | | FAVKGMEAARACVEILAAREKIAA | |
| I53-50B.1 | pentamer | NQHSHKDHETVRIAVVRARWHAEIVD | I53-50B: |
| SEQ ID NO: 15 | | ACVSAFEAAMRDIGGDRFAVDVFDVP | 24, 28, 36, 124, |
| | | GAYEIPLHARTLAETGRYGAVLGTAFV | 125, 127, 128, |
| | | VNGGIYRHEFVASAVIDGMMNVQLDT | 129, 131, |
| | | GVPVLSAVLTPHRYRDSDAHTLLFLAL | 132, 133, 135, |
| | | FAVKGMEAARACVEILAAREKIAA | 139 |
| I53-50B.1NegT2 | pentamer | NQHSHKDHETVRIAVVRARWHAEIVD | I53-50B: |
| SEQ ID NO: 16 | | ACVSAFEAAMRDIGGDRFAVDVFDVP | 24, 28, 36, 124, |
| | | GAYEIPLHARTLAETGRYGAVLGTAFV | 125, 127, 128, |
| | | VDGGIYDHEFVASAVIDGMMNVQLDT | 129, 131, 132, |
| | | GVPVLSAVLTPHEYEDSDADTLLFLAL | 133, 135, |
| | | FAVKGMEAARACVEILAAREKIAA | 139 |
| I53-50B.4PosT1 | trimer | NQHSHKDHETVRIAVVRARWHAEIVD | I53-50B: |
| SEQ ID NO: 17 | | ACVSAFEAAMRDIGGDRFAVDVFDVP | 24, 28, 36, 124, |
| | | GAYEIPLHARTLAETGRYGAVLGTAFV | 125, 127, 128, |
| | | VNGGIYRHEFVASAVINGMMNVQLNT | 129, 131, 132, |
| | | GVPVLSAVLTPHNYDKSKAHTLLFLAL | 133, 135, |
| | | FAVKGMEAARACVEILAAREKIAA | 139 |
| I53-50B.4PosT1 | trimer | MNQHSHKDHETVRIAVVRARWHAEIV | I53-50B: |
| SEQ ID NO: 27 | | DACVSAFEAAMRDIGGDRFAVDVFDV | 24, 28, 36, 124, |
| | | PGAYEIPLHARTLAETGRYGAVLGTAF | 125, 127, 128, |
| | | VVNGGIYRHEFVASAVINGMMNVQLN | 129, 131, |
| | | TGVPVLSAVLTPHNYDKSKAHTLLFLA | 132, 133, 135, |
| | | LFAVKGMEAARACVEILAAREKIAA | 139 |
| I53-50B genus | pentamer | NQHSHKD(Y/H)ETVRIAVVRARWHAEI | |
| SEQ ID NO: 18 | | VDACVSAFEAAM(A/R)DIGGDRFAVDV | |
| | | FDVPGAYEIPLHARTLAETGRYGAVLG | |
| | | TAFVV(N/D)GGIY(R/D)HEFVASAVI | |
| | | (D/N)GMMNVQL(S/D/N)TGVPVLSAVLTPH | |
| | | (R/E/N)Y(R/D/E)(D/K)S(D/K)A(H/D) | |
| | | TLLFLALFAVKGMEAARACVEILAAREKIAA | |
| I53_dn5A SEQ | | KYDGSKLRIGILHARWNAEIILALVLGA | |
| ID NO: 20 | | LKRLQEFGVKRENIIIETVPGSFELPYGS | |
| | | KLFVEKQKRLGKPLDAIIPIGVLIKGST | |
| | | MHFEYICDSTTHQLMKLNFELGIPVIFG | |
| | | VLTCLTDEQAEARAGLIEGKMHNHGE | |
| | | DWGAAAVEMATKFN | |
| I53_dn5B SEQ | | EEAELAYLLGELAYKLGEYRIAIRAYRI | |
| ID NO: 19 | | ALKRDPNNAEAWYNLGNAYYKQGRY | |
| | | REAIEYYQKALELDPNNAEAWYNLGN | |
| | | AYYERGEYEEAIEYYRKALRLDPNNAD | |
| | | AMQNLLNAKMREE | |

In some embodiments, the VLP comprises a fusion protein that has at least 95%, at least 96%, at least 97%, at least 98%, at least 99%, or 100% identity to any one of SEQ ID NO: 9-13 and comprises an RBD or coronavirus S protein as disclosed herein; and a second component that has at least 95%, at least 96%, at least 97%, at least 98%, at least 99%, or 100% identity to any one of SEQ ID NO: 13-18 or 27. In some embodiments, the VLP comprises a fusion protein that has at least 75% identity to any one of SEQ ID NO: 9-13 and comprises an RBD or coronavirus S protein as disclosed herein; and a second component that has at least 75% identity to any one of SEQ ID NO: 13-18 or 27. In some embodiments, the VLP comprises a fusion protein that has at least 95%, at least 96%, at least 97%, at least 98%, at least 99%, or 100% identity to SEQ ID NO: 19 and comprises an RBD or coronavirus S protein as disclosed herein; and a second component that has at least 95%, at least 96%, at least 97%, at least 98%, at least 99%, or 100% identity to SEQ ID NO: 20.

In some embodiments, the first component comprises the polypeptide sequence that has at least 95%, at least 96%, at least 97%, at least 98%, at least 99%, or 100% identity to any one of SEQ ID NOs: 1-6.

(SEQ ID NO: 1)
RFPNITNLCPFGEVENATRFASVYAWNRKRISNCVADYSVLYNSASFSTFKCYGVSPTKL
NDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGENCYFPLQSYGFQPTNGVGY
QPYRVVVLSFELLHAPATVCGPKKSTGGSGGSGSGGSGGSGSEKAAKAEEAARKMEEL
FKKHKIVAVLRANSVEEAIEKAVAVFAGGVHLIEITFTVPDADTVIKALSVLKEKGAIIGA
GTVTSVEQARKAVESGAEFIVSPHLDEEISQFAKEKGVFYMPGVMTPTELVKAMKLGHT
ILKLFPGEVVGPQFVKAMKGPFPNVKFVPTGGVNLDNVAEWFKAGVLAVGVGSALVK
GTPDEVREKAKAFVEKIRGATE
(SEQ ID NO: 2)
RFPNITNLCPFGEVENATRFASVYAWNRKRISNCVADYSVLYNSASFSTFKCYGVSPTKL
NDLCFTNVYADSFVIRGDEVRQIAPGQTGNIADYNYKLPDDFTGCVIAWNSNNLDSKVG
GNYNYLYRLFRKSNLKPFERDISTEIYQAGSTPCNGVKGFNCYFPLQSYGFQPTYGVGY
QPYRVVVLSFELLHAPATVCGPKKSTGGSGGSGSGGSGGSGSEKAAKAEEAARKMEEL
FKKHKIVAVLRANSVEEAIEKAVAVFAGGVHLIEITFTVPDADTVIKALSVLKEKGAIIGA
GTVTSVEQARKAVESGAEFIVSPHLDEEISQFAKEKGVFYMPGVMTPTELVKAMKLGHT
ILKLFPGEVVGPQFVKAMKGPFPNVKFVPTGGVNLDNVAEWFKAGVLAVGVGSALVK
GTPDEVREKAKAFVEKIRGATE
(SEQ ID NO: 3)
RFPNITNLCPFGEVFNATRFASVYAWNRKRISNCVADFSVLYNSASFSTFKCYGVSPTKL
NDLCWTNIYADSFVIRGDEVRQIAPGQTGKIADYNYKLPDDFTGCVIAWNSNNLDSKVG
GNYNYLYRLFRKSNLKPFERDISTEIYQAGSTPCNGVEGENCYFPLQSYGFQPTYGVGY
QPYRVVVLSFELLHAPATVCGPKKSTGGSGGSGSGGSGGSGSEKAAKAEEAARKMEEL
FKKHKIVAVLRANSVEEAIEKAVAVFAGGVHLIEITFTVPDADTVIKALSVLKEKGAIIGA
GTVTSVEQARKAVESGAEFIVSPHLDEEISQFAKEKGVFYMPGVMTPTELVKAMKLGHT
ILKLFPGEVVGPQFVKAMKGPFPNVKFVPTGGVNLDNVAEWFKAGVLAVGVGSALVK
GTPDEVREKAKAFVEKIRGATE
(SEQ ID NO: 4)
RFPNITNLCPFGEVFNATRFASVYAWNRKRISNCVADFSVLYNSASFSTFKCYGVSPTKL
NDLCWTNIYADSFVIRGDEVRQIAPGQTGNIADYNYKLPDDFTGCVIAWNSNNLDSKVG
GNYNYLYRLFRKSNLKPFERDISTEIYQAGSTPCNGVKGFNCYFPLQSYGFQPTYGVGY
QPYRVVVLSFELLHAPATVCGPKKSTGGSGGSGSGGSGGSGSEKAAKAEEAARKMEEL
FKKHKIVAVLRANSVEEAIEKAVAVFAGGVHLIEITFTVPDADTVIKALSVLKEKGAIIGA
GTVTSVEQARKAVESGAEFIVSPHLDEEISQFAKEKGVFYMPGVMTPTELVKAMKLGHT
ILKLFPGEVVGPQFVKAMKGPFPNVKFVPTGGVNLDNVAEWFKAGVLAVGVGSALVK
GTPDEVREKAKAFVEKIRGATE
(SEQ ID NO: 5)
RFPNITNLCPFGEVENATRFASVYAWNRKRISNCVADFSVLYNSASFSTFKCYGVSPTKL
NDLCWTNIYADSFVIRGDEVRQIAPGQTGKIADYNYKLPDDFTGCVIAWNSNNLDSKVG
GNYNYLYRLFRKSNLKPFERDISTEIYQAGSTPCNGVEGENCYFPLQSYGFQPTNGVGY
QPYRVVVLSFELLHAPATVCGPKKSTGGSGGSGSGGSGGSGSEKAAKAEEAARKMEEL
FKKHKIVAVLRANSVEEAIEKAVAVFAGGVHLIEITFTVPDADTVIKALSVLKEKGAIIGA
GTVTSVEQARKAVESGAEFIVSPHLDEEISQFAKEKGVFYMPGVMTPTELVKAMKLGHT
ILKLFPGEVVGPQFVKAMKGPFPNVKFVPTGGVNLDNVAEWFKAGVLAVGVGSALVK
GTPDEVREKAKAFVEKIRGATE
(SEQ ID NO: 6)
RFPNITNLCPFGEVENATRFASVYAWNRKRISNCVADFSVLYNSASFSTFKCYGVSPTKL
NDLCWTNIYADSFVIRGDEVRQIAPGQTGNIADYNYKLPDDFTGCVIAWNSNNLDSKVG
GNYNYLYRLFRKSNLKPFERDISTEIYQAGSTPCNGVKGFNCYFPLQSYGFQPTYGVGY
QPYRVVVLSFELLHAPATVCGPKKSTGGSGGSGSGGSGGSGSEKAAKAEEAARKMEEL
FKKHKIVAVLRANSVEEAIEKAVAVFAGGVHLIEITFTVPDADTVIKALSVLKEKGAIIGA
GTVTSVEQARKAVESGAEFIVSPHLDEEISQFAKEKGVFYMPGVMTPTELVKAMKLGHT
ILKLFPGEVVGPQFVKAMKGPFPNVKFVPTGGVNLDNVAEWFKAGVLAVGVGSALVK
GTPDEVREKAKAFVEKIRGATE
The amino acid sequence of the native or wild-type SARS-CoV-2 S
protein, subunit 1 is:
(SEQ ID NO: 7)
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMD
LEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSET
KCTLKSFTVEKGIYQTSNFRVQPTESIVRFPNITNLCPFGEVENATRFASVYAWNRKRISN
CVADYSVLYNSASFSTFKCYGVSPTKLNDLCFTNVYADSFVIRGDEVRQIAPGQTGKIA
DYNYKLPDDFTGCVIAWNSNNLDSKVGGNYNYLYRLFRKSNLKPFERDISTEIYQAGST
PCNGVEGENCYFPLQSYGFQPTNGVGYQPYRVVVLSFELLHAPATVCGPKKSTNLVKN
KCVNFNFNGLTGTGVLTESNKKFLPFQQFGRDIADTTDAVRDPQTLEILDITPCSFGGVS
VITPGTNTSNQVAVLYQDVNCTEVPVAIHADQLTPTWRVYSTGSNVFQTRAGCLIGAEH
VNNSYECDIPIGAGICASYQTQTNSPRRARSVASQSIIAYTMSLGAENSVAYSNNSIAIPT
NFTISVTTEILPVSMTKTSVDCTMYICGDSTECSNLLLQYGSFCTQLNRALTGIAVEQDK
NTQEVFAQVKQIYKTPPIKDFGGFNFSQILPDPSKPSKRSFIEDLLFNKVTLADAGFIKQY
GDCLGDIAARDLICAQKFNGLTVLPPLLTDEMIAQYTSALLAGTITSGWTFGAGAALQIP
FAMQMAYRFNGIGVTQNVLYENQKLIANQFNSAIGKIQDSLSSTASALGKLQDVVNQN
AQALNTLVKQLSSNFGAISSVLNDILSRLDKVEAEVQIDRLITGRLQSLQTYVTQQLIRAA.

The first component may comprise a receptor-binding domain of a coronavirus S protein. In some embodiments, the receptor-binding domain of a coronavirus S protein comprises the polypeptide sequence that has at least 95%, at least 96%, at least 97%, at least 98%, at least 99%, or 100% identity to any one of SEQ ID NOs: 21-24.

(SEQ ID NO: 21)
RFPNITNLCPFGEVFNATRFASVYAWNRKRISNCVADYSVLYNSASFST
FKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQ
AGSTPCNGVEGENCYFPLQSYGFQPTNGVGYQPYRVVVLSFELLHAPAT
VCGPKKST
(SEQ ID NO: 22)
RFPNITNLCPFGEVFNATRFASVYAWNRKRISNCVADYSVLYNSASFST
FKCYGVSPTKLNDLCFTNVYADSFVIRGDEVRQIAPGQTGNIADYNYKL
PDDFTGCVIAWNSNNLDSKVGGNYNYLYRLFRKSNLKPFERDISTEIYQ
AGSTPCNGVKGFNCYFPLQSYGFQPTYGVGYQPYRVVVLSFELLHAPAT
VCGPKKST
(SEQ ID NO: 23)
RFPNITNLCPFGEVFNATRFASVYAWNRKRISNCVADFSVLYNSASFST
FKCYGVSPTKLNDLCWTNIYADSFVIRGDEVRQIAPGQTGKIADYNYKL
PDDFTGCVIAWNSNNLDSKVGGNYNYLYRLFRKSNLKPFERDISTEIYQ
QAGSTPCNGVEGENCYFPLQSYGFQPTYGVGYPYRVVVLSFELLHAPAT
VCGPKKST
(SEQ ID NO: 24)
RFPNITNLCPFGEVFNATRFASVYAWNRKRISNCVADFSVLYNSASFST
FKCYGVSPTKLNDLCWTNIYADSFVIRGDEVRQIAPGQTGNIADYNYKL
PDDFTGCVIAWNSNNLDSKVGGNYNYLYRLFRKSNLKPFERDISTEIYQ
AGSTPCNGVKGFNCYFPLQSYGFQPTYGVGYQPYRVVVLSFELLHAPAT
VCGPKKST

In some embodiments, the first component comprises the polypeptide sequence that has at least 95%, at least 96%, at least 97%, at least 98%, at least 99%, or 100% identity to any one of SEQ ID NOs: 1-6 and further comprises a signal peptide. In some embodiments, the signal peptide comprises the sequence of SEQ ID NO: 25. In some embodiments, the signal peptide comprises the sequence of SEQ ID NO: 26.

(SEQ ID NO: 25)
MGILPSPGMPALLSLVSLLSVLLMGCVA
(SEQ ID NO: 26)
MGILPSPGMPALLSLVSLLSVLLMGCVAETGT

The polypeptides as described herein may have one of more amino acid substitutions from known variants of SARS-CoV2 (also called “variant strains of SARS-CoV2”). Such variant strains of SARS-CoV2 comprise mutations relative to the original strain of SARS-CoV2. The term “original” strain as used herein refers to the Wuhan strain of SARS-CoV-2 identified in 2019-2020. For example and without limitation, the polypeptides may comprise 1, 2, 3, 4, 5, 6, 7, or all 8 positions relative to SEQ ID NO: 7 selected from the group consisting of L18F, T20N, P26S, deletion of residues 69-70, D80A, D138Y, R190S, D215G, R346K, K417N, K417T, G446S, L452R, Y453F, S477N, T478I, T478K, V483A, E484K, E484Q, S494P, N501Y, A570D, D614G, H655Y, G669S, Q677H, P681H, P681R, A701V, T716L. The polypeptides may comprise one of the following naturally occurring mutations or combinations of mutations:
N501Y, optionally further including 1, 2, 3, 4, or 5 of deletion of one or both of residues 69-70, E484K, A570D, D614G, P681H, and/or T716L (UK variant);
K417N/E484K/N501Y, optionally further including 1, 2, 3, 4, or 5 of L18F, D80A, D215G, D614G, and/or A701V (South African variant);
K417N or T/E484K/N501Y, optionally further including 1, 2, 3, 4, or 5 of L18F, T20N, P26S, D138Y, R190S, D614G, and/or H655Y (Brazil variant);
L452R (Los Angeles variant);
L452R, T478K, E484Q, D614G, P681R (India variant);
E484K, D614G, Q677H (Nigeria variant);
E484K, N501Y, D614G, P681H (Philippines variant);
V483A, D614G, H655Y, G669S (France variant);
V367F, E484K, Q613H (UK variant);
R346K, E484K, N501Y, D614G, P681H (Colombia variant);
P384L, K417N, E484K, N501Y, D614G, A701V (South Africa variant);
L452R, N501Y, D614G, P681H (UK variant);
S494P, N501Y, D614G, P681H (UK variant);
L452R, D614G, Q677H (Egypt variant);
E484K, D614G, N679K, ins679GIAL (Russian variant);
E484K, D614G, A701V (USA variant);
L452R, D614G (USA variant);
S477N, D614G (USA variant);
E484K, D614G (Brazil variant);
T478K, D614G (Mexico variant);
N439K, E484K, D614G, P681H (UK variant);
K417N, E484K, N501Y, E516Q, D614G, A701V;
E484K, D614G, P681H;
Q414K, N450K, ins214TDR, D614G;
L452R, N501Y, A653V, H655Y;
E484K, N501T, H655Y;
L452R, D614G;
L452Q, F490S, D614G;
D614G, F490R, N394S. N501Y, P681H, R346S, Y449N, 137-145del (Congo variant, B.1640)
L452R, T478K, D614G, P681R (Delta variant); or
A67V, A69-70, T95I, G142D, A143-145, N211I, A212, ins215EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F (Omicron BA.1 variant);
T19I, LPPA24-27S, G142D, V213G, G339D, S371F, S373P, S375F, T376A, D405N, R408S, K417N, N440K, S477N, T478K, E484A, Q493R, Q498R, N501Y, Y505H, D614G, H655Y, N679K, P681H, N764K, D796Y, Q954H, N969K (Omicron BA.2 variant);
T19I, LPPA24-27S, G142D, V213G, G339D, S371F, S373P, S375F, T376A, D405N, R408S, K417N, N440K, L452Q, S477N, T478K, E484A, Q493R, Q498R, N501Y, Y505H, D614G, H655Y, N679K, P681H, S704L, N764K, D796Y, Q954H, N969K (Omicron BA.2.12.1 variant);
T19I, LPPA24-27S, Del 69-70, G142D, V213G, G339D, S371F, S373P, S375F, T376A, D405N, R408S, K417N, N440K, L452R, S477N, T478K, E484A, F486V, Q498R, N501Y, Y505H, D614G, H655Y, N679K, P681H, N764K, D796Y, Q954H, N969K (Omicron BA.4 variant);
T19I, LPPA24-27S, Del 69-70, G142D, V213G, G339D, S371F, S373P, S375F, T376A, D405N, R408S, K417N, N440K, L452R, S477N, T478K, E484A, F486V, Q498R, N501Y, Y505H, D614G, H655Y, N679K, P681H, N764K, D796Y, Q954H, N969K (Omicron BA.5 variant).

A polypeptide provided herein may comprise one or more conservative amino acid substitutions. The terminology “conservative amino acid substitution” is well known in the art, and relates to substitution of a particular amino acid by one having a similar characteristic (e.g., similar charge or hydrophobicity). Conservative mutations can include, without limitation, substitution of amino acid residues with e.g., similar charge or hydrophobicity but differing in size or bulkiness (e.g., to provide a cavity-filling function). A list of exemplary conservative amino acid substitutions is given in the table below.

| For Amino Acid | Code | Replace With |
|---|---|---|
| Alanine | A | D-ala, Gly, Aib, β-Ala, Acp, L-Cys, D-Cys |
| Arginine | R | D-Arg, Lys, D-Lys, homo-Arg, D-homo-Arg, Met, Ile, D-Met, D- |
| | | Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, Aib, β-Ala, Acp |
| Isoleucine | I | D-Ile, Val, D-Val, AdaA, AdaG, Leu, D-Leu, Met, D-Met |
| Leucine | L | D-Leu, Val, D-Val, AdaA, AdaG, Leu, D-Leu, Met, D-Met |
| Lysine | K | D-Lys, Arg, D-Arg, homo-Arg, D-homo-Arg, Met, D-Met, Ile, D- |
| | | Ile, Orn, D-Orn |
| Methionine | M | D-Met, S-Me-Cys, Ile, D-Ile, Leu, D-Leu, Val, D-Val |
| Phenylalanine | F | D-Phe, Tyr, D-Thr, L-Dopa, His, D-His, Trp, D-Trp, Trans-3,4 or |
| | | 5-phenylproline, AdaA, AdaG, cis-3,4 or 5-phenylproline, Bpa, D- |
| | | Bpa |
| Proline | P | D-Pro, L-I-thioazolidine-4-carboxylic acid, D-or-L-1-oxazolidine- |
| | | 4-carboxylic acid (Kauer, U.S. Pat. No. (4,511,390) |
| Serine | S | D-Ser, Thr, D-Thr, allo-Thr, Met, D-Met, Met (O), D-Met (O), L- |
| | | Cys, D-Cys |
| Threonine | T | D-Thr, Ser, D-Ser, allo-Thr, Met, D-Met, Met (O), D-Met (O), Val, |
| | | D-Val |
| Tyrosine | Y | D-Tyr, Phe, D-Phe, L-Dopa, His, D-His |
| Valine | V | D-Val, Leu, D-Leu, Ile, D-Ile, Met, D-Met, AdaA, AdaG |

Alternatively, a non-conservative amino acid substitution may be preferred, for example, when eradication of a flexible portion of the native coronavirus S protein secondary structure is desired, for example, by adding a cysteine residue (or vice versa). “Non-conservative substitution” refers to the substitution of an amino acid in one class with an amino acid from another class; for example, substitution of an Ala with Asp, Asn, Glu, or Gln. Additional non-limiting examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

### Nucleic Acids, Vectors, and Cells

In another aspect, the disclosure provides nucleic acids encoding a polypeptide or fusion protein of the disclosure. The nucleic acid sequence may comprise RNA (such as mRNA) or DNA. Such nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the proteins of the invention.

In another aspect, disclosure provides expression vectors comprising the isolated nucleic acid of any embodiment or combination of embodiments of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited to plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive).

In another aspect, the present disclosure provides cells comprising the polypeptide, the virus-like particle, the composition, the nucleic acid, and/or the expression vector of any embodiment or combination of embodiments of the disclosure, wherein the cells can be either prokaryotic or eukaryotic, such as mammalian cells. In some embodiments the cells may be transiently or stably transfected with the nucleic acids or expression vectors of the disclosure. Such transfection of expression vectors into prokaryotic and eukaryotic cells can be accomplished via any technique known in the art. A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide.

### Pharmaceutical Compositions

In another aspect, the disclosure provides pharmaceutical compositions/vaccines comprising
(a) the polypeptide, the virus-like particle, the composition, the nucleic acid, the expression vector, and/or the cell of embodiment or combination of embodiments herein; and
(b) a pharmaceutically acceptable carrier.

As shown in the examples that follow, the virus-like particles elicit potent and protective antibody responses against SARS-CoV-2. The virus-like particles of the disclosure induce neutralizing antibody titers roughly ten-fold higher than the prefusion-stabilized S ectodomain trimer despite a more than five-fold lower dose. Antibodies elicited by the virus-like particles target multiple distinct epitopes, suggesting that they may not be easily susceptible to escape mutations, and exhibit a significantly lower binding:neutralizing ratio than convalescent human sera, which may minimize the risk of vaccine-associated enhanced respiratory disease.

The compositions/vaccines may further comprise (a) a lyoprotectant; (b) a surfactant; (c) a bulking agent; (d) a tonicity adjusting agent; (e) a stabilizer; (f) a preservative and/or (g) a buffer. In some embodiments, the buffer in the pharmaceutical composition is a Tris buffer, a histidine buffer, a phosphate buffer, a citrate buffer or an acetate buffer. The composition may also include a lyoprotectant, e.g. sucrose, sorbitol or trehalose. In certain embodiments, the composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the nanostructure, in lyophilized or liquid form. Exemplary stabilizers include sucrose, sorbitol, glycine, inositol, sodium chloride, methionine, arginine, and arginine hydrochloride.

The virus-like particles may be the sole active agent in the composition, or the composition may further comprise one or more other agents suitable for an intended use, including but not limited to adjuvants to stimulate the immune system generally and improve immune responses overall. Any suitable adjuvant can be used. The term “adjuvant” refers to a compound or mixture that enhances the immune response to an antigen.

Exemplary types of adjuvants that may be used in a pharmaceutical composition provided herein include the following: 1. mineral-containing compositions; 2. oil emulsions; 3. saponin formulations; 4. virosomes and virus-like particles; 5. bacterial or microbial derivatives; 6. bioadhesives and mucoadhesives; 7. liposomes; 8. polyoxyethylene ether and polyoxyethylene ester formulations; 9. polyphosphazene (pcpp); 10. muramyl peptides; 11. imidazoquinolone compounds; 12. thiosemicarbazone compounds; 13. tryptanthrin compounds; 14. human immunomodulators; 15. lipopeptides; 16. benzonaphthyridines; 17. microparticles; 18. immunostimulatory polynucleotide (such as RNA or DNA; e.g., cpg-containing oligonucleotides).

Exemplary adjuvants that may be used in a pharmaceutical composition provided herein include, but are not limited to, 3M-052, Adju-Phos™, Adjumer™, albumin-heparin microparticles, Algal Glucan, Algammulin, Alum, Antigen Formulation, AS-2 adjuvant, ASO1, ASO3, autologous dendritic cells, autologous PBMC, Avridine™, B7-2, BAK, BAY R1005, Bupivacaine, Bupivacaine-HCl, BWZL, Calcitriol, Calcium Phosphate Gel, CCR5 peptides, CFA, Cholera holotoxin (CT) and Cholera toxin B subunit (CTB), Cholera toxin A1-subunit-Protein A D-fragment fusion protein, CpG, CPG-1018, CRL1005, Cytokine-containing Liposomes, D-Murapalmitine, DDA, DHEA, Diphtheria toxoid, DL-PGL, DMPC, DMPG, DOC/Alum Complex, Fowlpox, Freund's Complete Adjuvant, Gamma Inulin, Gerbu Adjuvant, GM-CSF, GMDP, hGM-CSF, hIL-12 (N222L), hTNF-alpha, IFA, IFN-gamma in pcDNA3, IL-12 DNA, IL-12 plasmid, IL-12/GMCSF plasmid (Sykes), IL-2 in pcDNA3, IL-2/Ig plasmid, IL-2/lg protein, IL-4, IL-4 in pcDNA3, Imiquimod™, ImmTher™, Immunoliposomes Containing Antibodies to Costimulatory Molecules, Interferon-gamma, Interleukin-1 beta, Interleukin-12, Interleukin-2, Interleukin-7, ISCOM(s)™, Iscoprep 7.0.3™, Keyhole Limpet Hemocyanin, Lipid-based Adjuvant, Liposomes, Loxoribine, LT(R192G), LT-OA or LT Oral Adjuvant, LT-R192G, LTK63, LTK72, Matrix-M™ adjuvant, MF59, MONTANIDE ISA 51, MONTANIDE ISA 720, MPL.TM., MPL-SE, MTP-PE, MTP-PE Liposomes, Murametide, Murapalmitine, NAGO, nCT native Cholera Toxin, Non-Ionic Surfactant Vesicles, non-toxic mutant E112K of Cholera Toxin mCT-E112K, p-Hydroxybenzoique acid methyl ester, pCIL-10, pCIL12, pCMVmCAT1, pCMVN, Peptomer-NP, Pleuran, PLG, PLGA, PGA, and PLA, Pluronic L121, PMMA, PODDS™, Poly rA: Poly rU, Polysorbate 80, Protein Cochleates, QS-21, Quadri A saponin, Quil-A, Rehydragel HPA, Rehydragel LV, RIBI, Ribilike adjuvant system (MPL, TMD, CWS), S-28463, SAF-1, Sclavo peptide, Sendai Proteoliposomes, Sendai-containing Lipid Matrices, Span 85, Specol, Squalane 1, Squalene 2, Stearyl Tyrosine, Tetanus toxoid (TT), Theramide™, Threonyl muramyl dipeptide (TMDP), Ty Particles, and Walter Reed Liposomes. Selection of an adjuvant depends on the subject to be treated. Preferably, a pharmaceutically acceptable adjuvant is used.

For example, the composition may include an aluminum salt adjuvant, an oil in water emulsion (e.g. an oil-in-water emulsion comprising squalene, such as MF59 or AS03), a TLR7 agonist (such as imidazoquinoline or imiquimod), or a combination thereof. In some embodiments, the adjuvant is a combination of an aluminum salt and CPG-1018. Suitable aluminum salts include hydroxides (e.g. oxyhydroxides), phosphates (e.g. hydroxyphosphates, orthophosphates), (e.g. see chapters 8 & 9 of Vaccine Design. (1995) eds. Powell & Newman. ISBN: 030644867X. Plenum), or mixtures thereof. The salts can take any suitable form (e.g. gel, crystalline, amorphous, etc.), with adsorption of antigen to the salt being an example. The concentration of Al⁺⁺⁺ in a composition for administration to a patient may be less than 5 mg/ml e.g. <4 mg/ml, <3 mg/ml, <2 mg/ml, <1 mg/ml, etc. A preferred range is between 0.3 and 1 mg/ml. A maximum of 0.85 mg/dose is preferred. Aluminum hydroxide and aluminum phosphate adjuvants are suitable for use with the disclosure.

In some embodiments, the composition including the virus-like particles may be the sole active agent in the composition, where no adjuvant is included, or wherein the composition is substantially free of an adjuvant. For example, no adjuvant may be added, or substance(s) having adjuvant property present but minimal quantities, such as quantities not expected to exert an adjuvant effect—for example, less than about 5%, less than about 4%, less than about 4%, less than about 3%, less than about 2%, less than about 1%, less than about 0.5%, less than about 0.1%, less than 5%, less than 4%, less than 4%, less than 3%, less than 2%, less than 1%, less than 0.5%, or less than 0.1% (w/v) of the pharmaceutical composition. In some embodiments, the composition including the virus-like particles may be the sole active agent in the composition and is free of an adjuvant, (e.g., Alum).

Also provided herein are unit doses of the pharmaceutical composition described herein. In some embodiments, the unit dose the unit dose comprises about 5 μg to about 10 μg, about 10 μg to about 15 μg, about 15 μg to about 20 μg, about 20 μg to about 30 μg, about 30 μg to about 40 μg, about 40 μg to about 50 μg, about 50 μg to about 60 μg, about 60 μg to about 70 μg, about 70 μg to about 80 μg, about 80 μg to about 90 μg, about 90 μg to about 100 μg, about 100 μg to about 110 μg, about 110 μg to about 120 μg, about 120 μg to about 130 μg, about 130 μg to about 140 μg, about 140 μg to about 150 μg, about 150 μg to about 200 μg, about 200 μg to about 250 μg, about 250 μg to about 300 μg, about 300 μg to about 350 μg, about 350 μg to about 400 μg, about 400 μg to about 450 μg, or about 450 μg to about 500 μg. In some embodiments, the unit dosage comprises 2 μg, 5 μg, 10 μg, 15 μg, 25 μg, 50 μg, 100 μg, or 125 μg of the protein complex. In some embodiments, the unit dosage comprises 5 μg of the protein complex. In some embodiments, the unit dosage comprises 25 μg of the protein complex. In some embodiments, the unit dosage comprises 125 μg of the protein complex. In some embodiments, the unit dosage comprises 100 μg of the protein complex.

In some embodiments, provided herein is a unit dose of the pharmaceutical composition described herein, wherein the unit dose comprises between about 25 μg and about 125 μg of the protein complex. In some embodiments, the unit dose of the pharmaceutical composition is between about 2 μg to about 125 μg, or between about 5 μg to about 125 g, or between about 15 μg to 125 μg, or between about 25 μg to about 125 μg, or between about 50 μg to about 125 μg, or between about 100 μg to about 125 μg of the protein complex.

The pH of the formulation can also vary. In general, it is between about pH 6.2 to about pH 8.0. In some embodiments, the pH is about 6.2, about 6.4, about 6.6, about 6.8, about 7.0, about 7.2, about 7.4, about 7.6, about 7.8, or about 8.0. Of course, the pH may also be within a range of values. Thus, in some embodiments the pH is between about 6.2 and about 8.0, between about 6.2 and 7.8, between about 6.2 and 7.6, between about 6.2 and 7.4, between about 6.2 and 7.2, between about 6.2 and 7.0, between about 6.2 and 6.8, between about 6.2 and about 6.6, or between about 6.2 and 6.4. In other embodiments, the pH is between 6.4 and about 8.0, between about 6.4 and 7.8, between about 6.4 and 7.6, between about 6.4 and 7.4, between about 6.4 and 7.2, between about 6.4 and 7.0, between about 6.4 and 6.8, or between about 6.4 and about 6.6. In still other embodiments, the pH is between about 6.6 and about 8.0, between about 6.6 and 7.8, between about 6.6 and 7.6, between about 6.6 and 7.4, between about 6.6 and 7.2, between about 6.6 and 7.0, or between about 6.6 and 6.8. In yet other embodiments, it is between about 6.8 and about 8.0, between about 6.8 and 7.8, between about 6.8 and 7.6, between about 6.8 and 7.4, between about 6.8 and 7.2, or between about 6.8 and 7.0. In still other embodiments, it is between about 7.0 and about 8.0, between about 7.0 and 7.8, between about 7.0 and 7.6, between about 7.0 and 7.4, between about 7.0 and 7.2, between about 7.2 and 8.0, between about 7.2 and 7.8, between about 7.2 and about 7.6, between about 7.2 and 7.4, between about 7.4 and about 8.0, about 7.4 and about 7.6, or between about 7.6 and about 8.0.

In some embodiments, the formulation can include one or more salts, such as sodium chloride, sodium phosphate, or a combination thereof. In general, each salt is present in the formulation at about 10 mM to about 200 mM. Thus, in some embodiments, any salt that is present is present at about 10 mM to about 200 mM, about 20 mM to about 200 mM, about 25 mM to about 200 mM, at about 30 mM to about 200 mM, at about 40 mM to about 200 mM, at about 50 mM to about 200 mM, at about 75 mM to about 200 mM, at about 100 mM to about 200 mM, at about 125 mM to about 200 mM, at about 150 mM to about 200 mM, or at about 175 mM to about 200 mM. In other embodiments, any salt that is present is present at about 10 mM to about 175 mM, about 20 mM to about 175 mM, about 25 mM to about 175 mM, at about 30 mM to about 175 mM, at about 40 mM to about 175 mM, at about 50 mM to about 175 mM, at about 75 mM to about 175 mM, at about 100 mM to about 175 mM, at about 125 mM to about 175 mM, or at about 150 mM to about 175 mM. In still other embodiments, any salt that is present is present at about 10 mM to about 150 mM, about 20 mM to about 150 mM, about 25 mM to about 150 mM, at about 30 mM to about 150 mM, at about 40 mM to about 150 mM, at about 50 mM to about 150 mM, at about 75 mM to about 150 mM, at about 100 mM to about 150 mM, or at about 125 mM to about 150 mM. In yet other embodiments, any salt that is present is present at about 10 mM to about 125 mM, about 20 mM to about 125 mM, about 25 mM to about 125 mM, at about 30 mM to about 125 mM, at about 40 mM to about 125 mM, at about 50 mM to about 125 mM, at about 75 mM to about 125 mM, or at about 100 mM to about 125 mM. In some embodiments, any salt that is present is present at about 10 mM to about 100 mM, about 20 mM to about 100 mM, about 25 mM to about 100 mM, at about 30 mM to about 100 mM, at about 40 mM to about 100 mM, at about 50 mM to about 100 mM, or at about 75 mM to about 100 mM. In yet other embodiments, any salt that is present is present at about 10 mM to about 75 mM, about 20 mM to about 75 mM, about 25 mM to about 75 mM, at about 30 mM to about 75 mM, at about 40 mM to about 75 mM, or at about 50 mM to about 75 mM. In still other embodiments, any salt that is present is present at about 10 mM to about 50 mM, about 20 mM to about 50 mM, about 25 mM to about 50 mM, at about 30 mM to about 50 mM, or at about 40 mM to about 50 mM. In other embodiments, any salt that is present is present at about 10 mM to about 40 mM, about 20 mM to about 40 mM, about 25 mM to about 40 mM, at about 30 mM to about 40 mM, at about 10 mM to about 30 mM, at about 20 mM to about 30, at about 25 mM to about 30 mM, at about 10 mM to about 25 mM, at about 20 mM to about 25 mM, or at about 10 mM to about 20 mM. In some embodiments, the sodium chloride is present in the formulation at about 100 mM. In some embodiments, the sodium phosphate is present in the formulation at about 25 mM.

Formulations comprising the mutated coronavirus proteins described herein may further comprise a solubilizing agent such as a nonionic detergent. Such detergents include, but are not limited to polysorbate 80 (Tween® 80), TritonX100 and polysorbate 20.

### Methods of Treatment

In another aspect, the disclosure provides methods to treat or limit development of a SARS-CoV-2 infection (e.g., infection with an original strain of SARS-CoV2 or infection with a variant strain of SARS-CoV2), comprising administering to a subject in need thereof an amount effective to treat or limit development of the infection of the polypeptide, virus-like particle, composition, nucleic acid, pharmaceutical composition, or vaccine of any embodiment herein (referred to as the “immunogenic composition”). The subject may be any suitable mammalian subject, including but not limited to a human subject.

Examples of variant strains of SARS-CoV2 have been detected around the world and include, without limitation: B.1.1.7 (UK), B.1.1.7+E484K (UK), B.1.351 (South Africa), P.1 (Brazil), B.1.617.2 (India), B.1.525 (Nigeria), B.1.427/B.1.429 (USA), P.3 (Philippines), B.1.616 (France), B.1.617.1 (India), B.1.617.3 (India), B.1.621 (Colombia), A.23.1+E484K (UK), C.37 (Peru), B.1.351+P384L (South Africa), B.1.1.7+L452R (UK), B.1.1.7+S494P (UK), C.36+L452R (Egypt), AT.1 (Russia), B.1.526 (USA), B.1.526.1 (USA), B.1.526.2 (USA), B.1.1.318, P.2 (Brazil), B.1.1.519 (Mexico), AV.1 (UK), B.1.620, B.1.351+E516Q, B.1.214.2, A.27, A.28, B.1.640 (Congo) C.16, B.617.2 (delta), and B.1.1.529 (omicron).

When the method comprises limiting a SARS-CoV-2 infection (e.g., infection with an original strain of SARS-CoV-2 or infection with a variant strain of SARS-CoV2), the immunogenic composition is administered prophylactically to a subject that is not known to be infected but may be at risk of exposure to SARS-CoV-2. As used herein, “limiting development” includes, but is not limited to accomplishing one or more of the following: (a) generating an immune response (antibody and/or cell-based, e.g., CD4 T cells, memory B cells, and/or CD8 T cells) to of SARS-CoV-2 in the subject; (b) generating neutralizing antibodies against SARS-CoV-2 in the subject (b) limiting build-up of SARS-CoV-2 titer in the subject after exposure to SARS-CoV-2; and/or (c) limiting or preventing development of SARS-CoV-2 symptoms after infection. The methods provided herein may be used to limit development of infection with an original strain of SARS-CoV2 and/or infection with a variant strain of SARS-CoV2. Exemplary symptoms of SARS-CoV-2 infection include, but are not limited to, fever, fatigue, cough, shortness of breath, chest pressure and/or pain, loss or diminution of the sense of smell, loss or diminution of the sense of taste, and respiratory issues including but not limited to pneumonia, bronchitis, severe acute respiratory syndrome (SARS), and upper and lower respiratory tract infections.

In some embodiments, the methods generate an immune response in a subject in the subject not known to be infected with SARS-CoV-2, wherein the immune response serves to limit development of infection and symptoms of a SARS-CoV-2 infection (e.g., infection with an original strain of SARS-CoV2 or infection with a variant strain of SARS-CoV2). In some embodiments, the immune response comprises generation of neutralizing antibodies and/or cell-based responses against SARS-CoV-2. In some embodiments, the immune response comprises generation of SARS-CoV-2 S protein or RBD antibody-specific responses with a mean geometric titer of at least 1×10⁵, at least 1×10⁶, at least 1×10⁷, at least 1×10⁸, or at least 1×10⁹ assay units. In an exemplary such embodiment, the immune response comprises generation of SARS-CoV-2 S protein or RBD antibody-specific responses with a mean geometric titer of at least 1×10⁵. In a further embodiment, the immune response comprises generation of antibodies against multiple antigenic epitopes.

As used herein, an “effective amount” refers to an amount of the immunogenic composition that is effective for treating and/or limiting SARS-CoV-2 infection (e.g., infection with an original strain of SARS-CoV2 or infection with a variant strain of SARS-CoV2). The polypeptide, virus-like particle, composition, nucleic acid, pharmaceutical composition, or vaccine of any embodiment herein are typically formulated as a pharmaceutical composition, such as those disclosed above, and can be administered via any suitable route, including orally, parentally, by inhalation spray, rectally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally. Polypeptide compositions may also be administered via microspheres, liposomes, immune-stimulating complexes (ISCOMs), or other microparticulate delivery systems or sustained release formulations introduced into suitable tissues (such as blood).

In another aspect, provided herein is a method of vaccinating a subject at risk of infection with SARS-CoV-2, comprising administering to the subject a pharmaceutical composition comprising an effective amount of the protein complex comprising a first component comprising three receptor-binding domain monomers of a coronavirus S protein and a first multimerization domain (e.g., a trimerization domain), and a second component comprising a second multimerization domain (e.g., a pentamerization domain); and one or more pharmaceutically acceptable diluents or excipients. In some embodiments, the pharmaceutical composition comprises an adjuvant. In some embodiments, the adjuvant is or comprises an oil. In some embodiments, the adjuvant is an oil-in-water (e.g., a squalene-in-water) emulsion. In some embodiments, the adjuvant is MF59®. In some embodiments, the adjuvant is an aluminum salt. In some embodiments, the adjuvant is CPG-1018. In some embodiments, the pharmaceutical composition comprises both an aluminum salt and CPG-1018. In some embodiments, the effective amount is 2 μg, 5 μg, 10 μg, 15 μg, 25 μg, 50 μg, 100 μg, or 125 μg of the protein complex. In some embodiments, the method comprises repeating the administering step.

In some embodiments, the method comprises administering a booster vaccine. In some embodiments, the subject is previously vaccinated with a SARS-CoV-2 vaccine and/or previously infected with SARS-CoV-2. In some embodiments, the subject has completed a full course of vaccination for SARS-CoV-2. In some embodiments, the subject has completed a full course of vaccination for an original strain vaccine of SARS-CoV-2.

In some embodiments, the subject has completed a partial course (e.g., has received one of two doses of a full course) of vaccination for an original strain of SARS-CoV-2. In some embodiments, the subject has received at least one dose of a vaccination for a variant strain of SARS-CoV-2 (e.g. a variant strain described herein). As used herein, the term “partial course” refers to a first administration of a series of two or more administrations constituting an art-recognized full course of vaccination. In some embodiments, the subject has received at least one dose of a vaccine comprising the receptor binding domain of a coronavirus S protein or a polynucleotide encoding the receptor binding domain of a coronavirus S protein. In some embodiments, the subject has received at least one dose of a vaccine comprising a coronavirus S protein or a polynucleotide encoding a coronavirus S protein. In some embodiments, the S protein is S2P. In some embodiments, the S protein is “HexaPro”—i.e., comprises the amino acid substitutions F817P, A892P, A899P, and A942P relative to a reference sequence.

In some embodiments, the method induces neutralizing antibody titers in the subject. In some embodiments, the method increases neutralizing antibody titers in the subject. In some embodiments, the method induces S protein-specific and RBD-specific IgG antibody titers in the subject. In some embodiments, the method induces cell mediated immunity (CD4 T cells, memory B cells, CD8 T cells) in the subject. In some embodiments, the method induces neutralizing antibody titers in the subject. In some embodiments, the method prevents infection with an original strain of SARS-CoV-2. In some embodiments, the method prevents infection with a variant strain of SARS-CoV-2. In some embodiments, the method reduces the severity of infection with an original strain of SARS-CoV-2. In some embodiments, the method reduces the severity of infection with a variant strain of SARS-CoV-2

In embodiments, the methods herein include vaccinating a subject at risk of infection with SARS-CoV-2, comprises administering the composition (e.g., a vaccine comprising a protein complex comprising a first component comprising a receptor-binding domain of a coronavirus S protein attached to a first multimerization domain, and a second component comprising a second multimerization domain; and one or more pharmaceutically acceptable diluents or excipients) to a subject that is at least 1 year old, 2 years old, 3 years old, 4 years old, 5 years old, 6 years old, 7 years old, 9 years old, 10 years old, 12 years old, 15 years old, 20 years old, 30 years old, 40 years old, 50 years old, 60 years old, 70 years old, 80 years old, or 90 years old. In some examples, the subject is an adult at least 18 years old. In some embodiments, the subject is an elderly adult that is at least 60 years old, or at least 70 years old, or at least 80 years old, or at least 90 years old. In some embodiments, the subject is a child from about 2 years old to about 18 years old, or from about 5 years old to about 18 years old, or from about 10 years old to about 18 years old. In some embodiments, the subject is an infant that is one year old, or 6 months old, or 3 months old. In some embodiments, the subject is a child under 5 years of age, or under 4 years of age, or under 3 years of age, or under 2 years of age, or under 1 year of age. In some embodiments, the subject is at least about 1 month old, about 2 months old, about 3 months old, about 4 months old, about 5 months old, about 6 months old, about 7 months old, about 8 months old, about 9 months old, about 10 months old, or about 11 months old. In some embodiments, the subject is at least about 1 to 8 weeks of age, or about 1 week to 12 weeks of age.

In another aspect, provided herein is a method of vaccinating a subject, comprising administering to the subject (i) a pharmaceutical composition comprising an effective amount of the protein complex comprising a first component comprising a receptor-binding domain of a coronavirus S protein and a first multimerization domain (e.g., a trimerization domain), and a second component comprising a second multimerization domain (e.g., a pentamerization domain). In some embodiments, the subject has received at least one dose of a vaccination for SARS-CoV-2 no less than 4 months before the administration of the protein complex. In some embodiments the subject has received at least one dose of a vaccination for SARS-CoV-2 no less than 3 months before the administration of the protein complex. In some embodiments the subject has received at least one dose of a vaccination for SARS-CoV-2 about 3 months to about 6 months before the administration of the protein complex. In some embodiments the subject has received at least one dose of a vaccination for SARS-CoV-2 3 months to 6 months before the administration of the protein complex. In some embodiments, the subject has received at least one dose of a vaccine comprising the receptor binding domain of a coronavirus S protein or a polynucleotide encoding the receptor binding domain of a coronavirus S protein. In some embodiments, the subject has received at least one dose of a vaccine comprising a coronavirus S protein or a polynucleotide encoding a coronavirus S protein. In some embodiments, the S protein is S2P or “HexaPro”—i.e., comprises the amino acid substitutions F817P, A892P, A899P, and A942P relative to a reference sequence. In some embodiments, the vaccine is an mRNA-based vaccine, an adenoviral vector-based vaccine, a protein-subunit based vaccine, or an inactivated virus vaccine. In some embodiments, the subject has completed a full course of vaccination for an original strain of SARS-CoV-2. In some embodiments, the subject has completed a partial course (e.g., has received one of two doses) of vaccination for an original strain of SARS-CoV-2. In some embodiments, the subject has previously been infected with SARS-CoV-2. In some embodiments, the subject has antibodies against SARS-CoV-2. In some embodiments, the subject has not previously been infected with SARS-CoV-2.

In some embodiments, the method induces neutralizing antibody titers in the subject. In some embodiments, the method induced SARS-CoV-2 ancestral strain specific neutralizing antibody titers in the subject. In some embodiments, the method induces a serum SARS-CoV-2 binding antibody response in the subject. In some embodiments, the antibody response is to a SARS-CoV-2 RBD and/or a SARS-CoV-2 S protein. In some embodiments, the method prevents infection with an original strain of SARS-CoV-2 and/or a variant strain of SARS-CoV-2. In some embodiments, the method reduces the severity of infection with coronavirus.

Dosage regimens can be adjusted to provide the optimum desired response (e.g., a therapeutic or prophylactic response). A suitable dosage range may, for instance, be 0.1 μg/kg to 0.5 μg/kg body weight, 0.5 μg/kg to 1 μg body weight, 1 μg/kg to 2 μg/kg body weight, 2 μg/kg to 3 μg/kg body weight, 3 μg/kg to 4 μg/kg body weight, 4 μg/kg to 5 μg/kg body weight, 5 μg/kg to 6 μg/kg body weight, 6 μg/kg to 7 μg/kg body weight, 7 μg/kg to 8 μg/kg body weight, 8 μg/kg to 9 μg/kg body weight, 9 μg/kg to 10 μg/kg body weight, 10 μg/kg to 15 μg/kg body weight, 15 μg/kg to 20 μg/kg body weight, 20 μg/kg to 25 μg/kg body weight, 25 μg/kg to 30 μg/kg body weight, 30 μg/kg to 35 μg/kg body weight, 35 μg/kg to 40 μg/kg body weight, 40 μg/kg to 45 μg/kg body weight, 45 μg/kg to 50 μg/kg body weight, 50 μg/kg to 55 μg/kg body weight, 55 μg/kg to 60 μg/kg body weight, 60 μg/kg to 65 μg/kg body weight, 65 μg/kg to 70 μg/kg body weight, 70 μg/kg to 75 μg/kg body weight, 75 μg/kg to 80 μg/kg body weight, 80 μg/kg to 85 μg/kg body weight, 85 μg/kg to 90 μg/kg body weight, 90 μg/kg to 95 μg/kg body weight, 95 μg/kg to 100 μg/kg body weight, 100 μg/kg to 150 μg body weight, 150 μg/kg to 200 μg body weight, 200 μg/kg to 250 μg/kg body weight, 250 μg/kg to 300 μg/kg body weight, 300 μg/kg to 350 μg/kg body weight, 350 μg/kg to 400 μg/kg body weight, 400 μg/kg to 450 μg/kg body weight, 450 μg/kg to 500 μg body weight, 500 μg/kg to 550 μg body weight, 550 μg/kg to 600 μg body weight, 600 μg/kg to 650 μg body weight, 650 μg/kg to 700 μg body weight, 700 μg/kg to 750 μg/kg body weight, 750 μg/kg to 800 μg/kg body weight, 800 μg/kg to 850 μg/kg body weight, 850 μg/kg to 900 μg/kg body weight, 900 μg/kg to 950 μg/kg body weight, 950 μg/kg to 1 mg/kg body weight, 1 mg/kg to 2 mg/kg body weight, 2 mg/kg to 3 mg/kg body weight, 3 mg/kg to 4 mg/kg body weight, 4 mg/kg to 5 mg/kg body weight, 5 mg/kg to 6 mg/kg body weight, 6 mg/kg to 7 mg/kg body weight, 7 mg/kg to 8 mg/kg body weight, 8 mg/kg to 90 mg/kg body weight, 90 mg/kg to 100 mg/kg body weight, 100 mg/kg to 150 mg/kg body weight, 150 mg/kg to 200 mg/kg body weight, 200 mg/kg to 250 mg/kg body weight, 250 mg/kg to 300 mg/kg body weight, 300 mg/kg to 350 mg/kg body weight, 350 mg/kg to 400 mg/kg body weight, 400 mg/kg to 450 mg/kg body weight, 450 mg/kg to 500 mg/kg body weight, 500 mg/kg to 550 mg/kg body weight, 550 mg/kg to 600 mg/kg body weight, 600 mg/kg to 650 mg/kg body weight, 650 mg/kg to 700 mg/kg body weight, 700 mg/kg to 750 mg/kg body weight, 750 mg/kg to 800 mg/kg body weight, 800 mg/kg to 850 mg/kg body weight, 850 mg/kg to 900 mg/kg body weight, 900 mg/kg to 950 mg/kg body weight, or 950 mg/kg to 1 g/kg of the protein complex.

The composition can be delivered in a single bolus, or may be administered more than once (e.g., 2, 3, 4, 5, or more times) as determined by attending medical personnel.

In some embodiments, about 1 μg, about 2 μg, about 3 μg, about 4 μg, about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 100 μg, about 125 μg, about 150 μg, about 175 μg, about 200 μg, about 225 μg, about 250 μg, about 275 μg, about 300 μg, about 325 μg, bout 350 μg, about 375 μg, about 400 μg, about 425 μg, about 450 μg, about 475 μg, or about 500 μg of the protein complex are administered. In some embodiments, about 5 μg to about 10 μg, about 10 μg to about 15 μg, about 15 μg to about 20 μg, about 20 μg to about 30 μg, about 30 μg to about 40 μg, about 40 μg to about 50 μg, about 50 μg to about 60 μg, about 60 μg to about 70 μg, about 70 μg to about 80 μg, about 80 μg to about 90 μg, about 90 μg to about 100 μg, about 100 μg to about 110 μg, about 110 μg to about 120 μg, about 120 μg to about 130 μg, about 130 μg to about 140 μg, about 140 μg to about 150 μg, about 150 μg to about 200 μg, about 200 μg to about 250 μg, about 250 μg to about 300 μg, about 300 μg to about 350 μg, about 350 μg to about 400 μg, about 400 μg to about 450 μg, or about 450 μg to about 500 μg of the protein complex are administered.

In some embodiments, about 10 μg to about 100 μg, about 10 μg to about 150 μg, about 10 μg to about 200 μg, about 10 μg to about 250 μg, about 10 μg to about 300 μg, about 10 μg to about 350 μg, about 10 μg to about 400 μg, about 10 μg to about 450 μg, or about 10 μg to about 500 μg of the protein complex are administered.

In some embodiments, about 25 μg to about 100 μg, about 25 μg to about 150 μg, about 25 μg to about 200 μg, about 25 μg to about 250 μg, about 25 μg to about 300 μg, about 25 μg to about 350 μg, about 25 μg to about 400 μg, about 25 μg to about 450 μg, or about 25 μg to about 500 μg of the protein complex are administered.

In some embodiments, about 50 μg to about 100 μg, about 50 μg to about 150 μg, about 50 μg to about 200 μg, about 50 μg to about 250 μg, about 50 μg to about 300 μg, about 50 μg to about 350 μg, about 50 μg to about 400 μg, about 50 μg to about 450 μg, or about 50 μg to about 500 μg of the protein complex are administered.

In some embodiments, about 5 μg to about 150 μg, about 10 μg to about 150 μg, about 25 μg to about 150 μg, about 50 μg to about 150 μg, about 75 μg to about 150 μg, about 100 μg to about 150 μg, or about 125 μg to about 150 μg of the protein complex are administered.

In some embodiments, about 5 μg to about 125 μg, about 10 μg to about 125 μg, about 25 μg to about 125 μg, about 50 μg to about 125 μg, about 75 μg to about 125 μg, or about 100 μg to about 125 μg of the protein complex are administered.

In some embodiments, about 5 μg to about 100 μg, about 10 μg to about 100 μg, about 25 μg to about 100 μg, about 50 μg to about 100 μg, or about 75 μg to about 100 μg of the protein complex are administered.

In some embodiments, about 5 μg to about 75 μg, about 10 μg to about 75 μg, about 25 μg to about 75 μg, or about 50 μg to about 75 μg of the protein complex are administered.

In some embodiments, about 5 μg to about 50 μg, about 10 μg to about 50 μg, or about 25 μg to about 50 μg of the protein complex are administered.

The dose amount described herein can be converted to molar amounts or adjusted, depending on the molecular mass of the protein complex, to deliver the same or similar molar amounts of the antigen (RBD). The protein complexes of the disclosure generally have molecular masses of about 4 MDa (60 copies each of 50 kDa CompA-RBD and 17 kDa CompB). The RBDs of the disclosure generally have molecular masses of about 23 kDa. Accordingly, 100 μg of a protein complex may be about 2.5 picomoles (pmol), and each 100 μg of protein complex may include about 34 μg of the RBD.

In some embodiments, about 1 μg, about 2 μg, about 3 μg, about 4 μg, about 5 μg, about 1 pmol, about 15 μg, about 2 pmol, about 25 μg, about 3 pmol, about 35 μg, about 4 pmol, about 45 μg, about 5 pmol, about 55 μg, about 6 pmol, about 65 μg, about 7 pmol, about 75 μg, about 8 pmol, about 85 μg, about 9 pmol, about 10 pmol, about 125 μg, about 15 pmol, about 175 μg, about 20 pmol, about 225 μg, about 25 pmol, about 275 μg, about 30 pmol, about 325 μg, bout 35 pmol, about 375 μg, about 40 pmol, about 425 μg, about 45 pmol, about 475 μg, or about 50 pmol of the protein complex are administered.

In some embodiments, about 0.25 pmol to about 10 pmol, about 0.25 pmol to about 15 pmol, about 0.25 pmol to about 20 pmol, about 0.25 pmol to about 25 pmol, about 0.25 pmol to about 30 pmol, about 0.25 pmol to about 35 pmol, about 0.25 pmol to about 40 pmol, about 0.25 pmol to about 45 pmol, or about 0.25 pmol to about 50 pmol of the protein complex are administered.

In some embodiments, about 0.5 pmol to about 10 pmol, about 0.5 pmol to about 15 pmol, about 0.5 pmol to about 20 pmol, about 0.5 pmol to about 25 pmol, about 0.5 pmol to about 30 pmol, about 0.5 pmol to about 35 pmol, about 0.5 pmol to about 40 pmol, about 0.5 pmol to about 45 pmol, or about 0.5 pmol to about 50 pmol of the protein complex are administered.

In some embodiments, about 1 pmol to about 10 pmol, about 1 pmol to about 15 pmol, about 1 pmol to about 20 pmol, about 1 pmol to about 25 pmol, about 1 pmol to about 30 pmol, about 1 pmol to about 35 pmol, about 1 pmol to about 40 pmol, about 1 pmol to about 45 pmol, or about 1 pmol to about 50 pmol of the protein complex are administered.

In some embodiments, about 2 pmol to about 10 pmol, about 2 pmol to about 15 pmol, about 2 pmol to about 20 pmol, about 2 pmol to about 25 pmol, about 2 pmol to about 30 pmol, about 2 pmol to about 35 pmol, about 2 pmol to about 40 pmol, about 2 pmol to about 45 pmol, or about 2 pmol to about 50 pmol of the protein complex are administered.

In some embodiments, about 5 pmol to about 10 pmol, about 5 pmol to about 15 pmol, about 5 pmol to about 20 pmol, about 5 pmol to about 25 pmol, about 5 pmol to about 30 pmol, about 5 pmol to about 35 pmol, about 5 pmol to about 40 pmol, about 5 pmol to about 45 pmol, or about 5 pmol to about 50 pmol of the protein complex are administered.

In some embodiments, about 0.5 pmol to about 15 pmol, about 1 pmol to about 15 pmol, about 2 pmol to about 15 pmol, about 5 pmol to about 15 pmol, about 7 pmol to about 15 pmol, about 10 pmol to about 15 pmol, or about 12 pmol to about 15 pmol of the protein complex are administered.

In some embodiments, about 0.5 pmol to about 12 pmol, about 1 pmol to about 12 pmol, about 2 pmol to about 12 pmol, about 5 pmol to about 12 pmol, about 7 pmol to about 12 pmol, or about 10 pmol to about 12 pmol of the protein complex are administered.

In some embodiments, about 0.5 pmol to about 10 pmol, about 1 pmol to about 10 pmol, about 2 pmol to about 10 pmol, about 5 pmol to about 10 pmol, or about 7 pmol to about 10 pmol of the protein complex are administered.

In some embodiments, about 0.5 pmol to about 7 pmol, about 1 pmol to about 7 pmol, about 2 pmol to about 7 pmol, or about 5 pmol to about 7 pmol of the protein complex are administered.

In some embodiments, about 0.5 pmol to about 5 pmol, about 1 pmol to about 5 pmol, or about 2 pmol to about 5 pmol of the protein complex are administered.

In some embodiments, about 10 μg to about 100 μg, about 10 μg to about 150 μg, about 10 μg to about 200 μg, about 10 μg to about 250 μg, about 10 μg to about 300 μg, about 10 μg to about 350 μg, about 10 μg to about 400 μg, about 10 μg to about 450 μg, or about 10 μg to about 500 μg of the RBD are administered.

In some embodiments, about 25 μg to about 100 μg, about 25 μg to about 150 μg, about 25 μg to about 200 μg, about 25 μg to about 250 μg, about 25 μg to about 300 μg, about 25 μg to about 350 μg, about 25 μg to about 400 μg, about 25 μg to about 450 μg, or about 25 μg to about 500 μg of the RBD are administered.

In some embodiments, about 50 μg to about 100 μg, about 50 μg to about 150 μg, about 50 μg to about 200 μg, about 50 μg to about 250 μg, about 50 μg to about 300 μg, about 50 μg to about 350 μg, about 50 μg to about 400 μg, about 50 μg to about 450 μg, or about 50 μg to about 500 μg of the RBD are administered.

In some embodiments, about 5 μg to about 150 μg, about 10 μg to about 150 μg, about 25 μg to about 150 μg, about 50 μg to about 150 μg, about 75 μg to about 150 μg, about 100 μg to about 150 μg, or about 125 μg to about 150 μg of the RBD are administered.

In some embodiments, about 5 μg to about 125 μg, about 10 μg to about 125 μg, about 25 μg to about 125 μg, about 50 μg to about 125 μg, about 75 μg to about 125 μg, or about 100 μg to about 125 μg of the RBD are administered.

In some embodiments, about 5 μg to about 100 μg, about 10 μg to about 100 μg, about 25 μg to about 100 μg, about 50 μg to about 100 μg, or about 75 μg to about 100 μg of the RBD are administered.

In some embodiments, about 5 μg to about 75 μg, about 10 μg to about 75 μg, about 25 μg to about 75 μg, or about 50 μg to about 75 μg of the RBD are administered.

In some embodiments, about 5 μg to about 50 μg, about 10 μg to about 50 μg, or about 25 μg to about 50 μg of the RBD are administered.

In some embodiments, about 25 μg to about 125 of the protein complex is administered. In some embodiments, about 25 μg to about 100 of the protein complex is administered.

In some embodiments, about 10 μg to about 125 of the protein complex is administered. In some embodiments, about 10 μg to about 100 of the protein complex is administered.

In some embodiments, about 25 μg to about 125 of the protein complex is administered without an adjuvant. In some embodiments, about 25 μg to about 100 of the protein complex is administered without an adjuvant.

In some embodiments, about 10 μg to about 125 of the protein complex is administered without an adjuvant. In some embodiments, about 10 μg to about 100 of the protein complex is administered without an adjuvant.

Protein complexes and pharmaceutical compositions thereof may be administered on a single dose schedule or a multiple dose schedule. Multiple doses may be used in a primary immunization schedule. In a multiple dose schedule, the various doses may be given by the same or different routes e.g., a parenteral prime and mucosal boost, a mucosal prime and parenteral boost, etc. In some embodiments, the second dose of a multiple dose regimen is administered about 2 weeks, about 3 weeks, about 4 weeks, about 5 weeks, or about 6 weeks after the prior dose. In embodiments, the each subsequent dose is administered 3 weeks after administration of the prior dose. In embodiments, the first dose is administered at day 0, and the second dose is administered at day 21. In embodiments, the first dose is administered at day 0, and the second dose is administered at day 28.

Multiple doses of the boost may be used in a heterologous boost immunization schedule. For example, one or more doses of a primary vaccine may be administered followed by more than one administrations of the boost vaccine. In a multiple dose boost schedule, the various boost doses may be given by the same or different routes e.g., a parenteral prime and mucosal boost, a mucosal prime and parenteral boost, etc. In some embodiments, the second dose of a multiple dose boost regimen is administered about 2 weeks, about 3 weeks, about 4 weeks, about 5 weeks, or about 6 weeks after the prior dose. In some embodiments, each subsequent dose is administered 3 weeks after administration of the prior dose. In some embodiments, the first boost dose is administered at day 0, and the second boost dose is administered at day 21. In some embodiments, the first boost dose is administered at day 0, and the second boost dose is administered at day 28. In some embodiments, the first boost dose is administered at day 0, and the second boost dose is administered at 3 months

In some embodiments, a method comprises administering a first dose and a second dose of the pharmaceutical composition, wherein the second dose is administered about 2 weeks to about 12 weeks, or about 4 weeks to about 12 weeks after the first dose is administered. In various further embodiments, the second dose is administered about 2, 3, 4, 5, 6, 7, 8, 9, 10, 11, or 12 weeks, about 1 month, about 2 months, about 3 months, about 4 months, about 5 months, about 6 months, about 9 months, about 12 months, about 18 months, about 2 years, about 3 years, about 4 years, or about 5 years after the first dose. In another embodiment, three doses may be administered, with a second dose administered about 1, 2, 3, 4, 5, 6, 7, 8, 9, 10, 11, or 12 weeks, about 1 month, about 2 months, about 3 months, about 4 months, about 5 months, about 6 months, about 9 months, about 12 months, about 18 months, about 2 years, about 3 years, about 4 years, or about 5 years after the first dose, and the third dose administered about 1, 2, 3, 4, 5, 6, 7, 8, 9, 10, 11, or 12 weeks, about 1 month, about 2 months, about 3 months, about 4 months, about 5 months, about 6 months, about 9 months, about 12 months, about 18 months, about 2 years, about 3 years, about 4 years, or about 5 years after the second dose.

The protein complexes and pharmaceutical compositions of the disclosure may also be used for heterologous prime-boost vaccination. In some embodiments, a method comprises administering a protein complex or pharmaceutical composition thereof about 2 weeks to about 12 weeks, or about 4 weeks to about 12 weeks after another vaccine, such as a heterologous prime vaccine. In further embodiments, the pharmaceutical composition is administered about 2, 3, 4, 5, 6, 7, 8, 9, 10, 11, or 12 weeks, about 1 month, about 2 months, about 3 months, about 4 months, about 5 months, about 6 months, about 9 months, about 12 months, about 18 months, about 2 years, about 3 years, about 4 years, or about 5 years after the other vaccine. In further embodiments, the protein complex or pharmaceutical composition thereof is administered about 2 or more months, about 3 or more months, about 4 or more months, about 5 or more months, about 6 or more months, about 8 or more months, about 10 or more months, or about 12 or months after an earlier vaccine. In some embodiments, a method comprises administering a protein complex or pharmaceutical composition thereof about 2 months to about 8 months, or about 2 months to about 6 months after another vaccine. The interval between first (prime) vaccine and second (boost) vaccine may be 1, 2, 3, 4, 5, 6, 7, 8, 9, 10, 11, or 12 months, or any other suitable interval. The prime vaccine may include multiple doses of the same vaccine, and the heterologous boost vaccine may include multiple doses of the same heterologous vaccine, administered at suitable intervals.

In variations, the method may comprise administering a protein complex or pharmaceutical composition thereof indefinitely, e.g., over regular intervals. For example, the regular intervals may include every 3 months, every 6 months, every 12 months, every 18 months, or every 24 months. In some embodiments, the polypeptide sequence of the antigen may be modified to compensate for antigenic drift.

The protein complexes and pharmaceutical compositions of the disclosure may also be used for homologous prime-boost vaccination (e.g., administering a booster dose following a primary regimen of the same vaccine). In some embodiments, a method comprises administering a protein complex or pharmaceutical composition thereof about 2 weeks to about 12 weeks, or about 4 weeks to about 12 weeks after another vaccine, such as a heterologous prime vaccine. In further embodiments, the pharmaceutical composition is administered about 2, 3, 4, 5, 6, 7, 8, 9, 10, 11, or 12 weeks, about 1 month, about 2 months, about 3 months, about 4 months, about 5 months, about 6 months, about 9 months, about 12 months, about 18 months, about 2 years, about 3 years, about 4 years, or about 5 years after the other vaccine. In further embodiments, the protein complex or pharmaceutical composition thereof is administered about 2 or more months, about 3 or more months, about 4 or more months, about 5 or more months, about 6 or more months, about 8 or more months, about 10 or more months, or about 12 or months after an earlier vaccine. In some embodiments, a method comprises administering a protein complex or pharmaceutical composition thereof about 2 months to about 8 months, or about 2 months to about 6 months after another vaccine. The interval between first (prime) vaccine and second (boost) vaccine may be 1, 2, 3, 4, 5, 6, 7, 8, 9, 10, 11, or 12 months, or any other suitable interval. The prime vaccine may include multiple doses of the same vaccine, and the homologous boost vaccine may include multiple doses of the homologous vaccine, administered at suitable intervals. In some embodiments, the method comprises administering a protein complex or pharmaceutical composition thereof continuously, e.g., over regular intervals. For example, the regular intervals may include every 3 months, every 6 months, every 12 months, every 18 months, or every 24 months

The disclosure further provides prime-boost strategies that employ any known or subsequently developed vaccine—including but not limited to a protein, DNA, RNA, inactivated virus, or viral vector vaccine—together with a protein complex or pharmaceutical composition as described herein. For example, the protein complexes described herein may be used as a primary vaccine followed by heterologous boost with another vaccine. Optionally, the subject may receive a further vaccination with a protein complex described herein. In other variations, another vaccine is used as the primary vaccine and a protein complex described herein is administered one or more times to boost the response to the primary vaccine.

Suitable vaccines for use as primary vaccines or as heterologous boost vaccines may include those marketed for use in humans by Moderna®, Pfizer®/BioNTech®, AstraZeneca®, Johnson & Johnson®, Novavax®, Sanofi®, SK Biosciences®, Medicago®, and Bavarian Nordic®. The protein complexes and pharmaceutical compositions described herein may be used in heterologous vaccination strategies with these and other vaccines for SARS-CoV-2.

In various other embodiments of prime-boost dosing, the administering comprises
(a) administering a prime dose to the subject of a protein (e.g., a subunit vaccine), DNA, mRNA, inactivated virus, or adenoviral vector vaccine, wherein the protein, DNA, mRNA, inactivated virus, or adenoviral vector vaccine comprising or encoding a coronavirus S protein or antigenic fragment thereof; and
(b) administering a boost dose to the subject of the polypeptide, virus-like particle, composition, nucleic acid, pharmaceutical composition, or vaccine of any embodiment or combination disclosed herein.

In an alternative embodiment, the administering comprises
(a) administering a prime dose of any embodiment or combination disclosed herein to the subject; and
(b) administering a boost dose to the subject of a protein (e.g., a subunit vaccine), DNA, mRNA, inactivated virus, or adenoviral vector vaccine, wherein the protein DNA, mRNA, inactivated virus, or adenoviral vector vaccine comprising or encoding a coronavirus S protein or antigenic fragment thereof.

In either of these embodiments, any suitable protein (e.g., a subunit vaccine), DNA, mRNA, inactivated virus, adenoviral vector vaccine, or protein-based vaccine may be used in conjunction with the immunogenic compositions of the present disclosure, including but not limited to vaccines to be developed as well as those available from Moderna®, Pfizer®/BioNTech®, AstraZeneca®, Johnson & Johnson®, Novavax®, Sanofi®, SK Biosciences®, Medicago®, and Bavarian Nordic®, etc.

In some embodiments, the administering comprises
(a) administering a prime dose of any embodiment or combination disclosed herein to the subject; and
(b) administering a boost dose to the subject of a protein (e.g., a subunit vaccine), DNA, mRNA, or adenoviral vector vaccine which is authorized for use to limiting SARS-CoV2 infection (e.g., any suitable DNA, mRNA, inactivated virus, adenoviral vector, or protein-based vaccine, including those available from Moderna®, Pfizer®/BioNTech®, AstraZeneca®, Johnson & Johnson®, Novavax®, Sanofi®, SK Biosciences®, Medicago®, and Bavarian Nordic®); and
(b) administering a boost dose of any embodiment or combination disclosed herein to the subject.

In some embodiments, the administering comprises
(a) administering to the subject a full course of a protein (e.g., a subunit vaccine), DNA, mRNA, inactivated virus, or adenoviral vector vaccine which is authorized for use to limiting SARS-CoV2 infection (e.g., any suitable DNA, mRNA, inactivated virus, adenoviral vector, or protein-based vaccine); and
(b) administering an effective amount (i.e., a boost dose) of any embodiment or combination disclosed herein to the subject.

In another embodiment of the methods, the subject is infected with a severe acute respiratory (SARS) virus, including but not limited to SARS-CoV-2, wherein the administering elicits an immune response against the SARS virus in the subject that treats a SARS virus infection in the subject. When the method comprises treating a SARS-CoV-2 infection, the immunogenic compositions are administered to a subject that has already been infected with SARS-CoV-2, and/or who is suffering from symptoms (as described above) indicating that the subject is likely to have been infected with SARS-CoV-2.

In some embodiments, the administering comprises
(a) administering an effective amount (i.e., a prime dose) of any embodiment or combination disclosed herein to the subject; and
(b) administering to the subject a full course of a protein (e.g., a subunit vaccine), DNA, mRNA, inactivated virus, or adenoviral vector vaccine which is authorized for use to limiting SARS-CoV2 infection (e.g., any suitable DNA, mRNA, inactivated virus, adenoviral vector, or protein-based vaccine).

In another embodiment of the methods, the subject is infected with a severe acute respiratory (SARS) virus, including but not limited to SARS-CoV-2, wherein the administering elicits an immune response against the SARS virus in the subject that treats a SARS virus infection in the subject. When the method comprises treating a SARS-CoV-2 infection, the immunogenic compositions are administered to a subject that has already been infected with SARS-CoV-2, and/or who is suffering from symptoms (as described above) indicating that the subject is likely to have been infected with SARS-CoV-2.

In some embodiments, the subject has received one or more doses of a DNA, inactivated virus, mRNA, adenoviral vector, or protein-based vaccine which is authorized for use to limit SARS-CoV2 infection (e.g., any suitable DNA, mRNA, inactivated virus, adenoviral vector, or protein-based vaccine). In some embodiments, the subject has received a single dose of a DNA, mRNA, inactivated virus, adenoviral vector, or protein-based vaccine which is authorized for use to limit SARS-CoV2 infection (e.g., any suitable DNA, mRNA, inactivated virus, adenoviral vector, or protein-based vaccine). In some embodiments, the subject has received two doses of a DNA, mRNA, inactivated virus, adenoviral vector, or protein-based vaccine which is authorized for use to limiting SARS-CoV2 infection (e.g., any suitable DNA, mRNA, inactivated virus, adenoviral vector vaccine, or protein-based vaccine). In some embodiments, the subject has received a full course of a DNA, mRNA, inactivated virus, adenoviral vector, or protein-based vaccine which is authorized for use to limiting SARS-CoV2 infection (e.g., any suitable DNA, mRNA, inactivated virus, adenoviral vector, or protein-based vaccine).

In some embodiments, the DNA, mRNA, inactivated virus, adenoviral vector, or protein-based vaccine is a vaccine against an original strain of SARS-CoV2. In some embodiments, the DNA, mRNA, inactivated virus, adenoviral vector, or protein-based vaccine is a vaccine against a variant strain of SARS-CoV2.

In some embodiments, a subject has received at least one dose of a vaccine comprising the receptor binding domain of a coronavirus S protein or a polynucleotide encoding the receptor binding domain of a coronavirus S protein prior to receiving a dose or boost of any embodiment or combination disclosed herein. In some embodiments, a subject has received at least one dose of a vaccine comprising a coronavirus S protein or a polynucleotide encoding a coronavirus S protein prior to receiving a dose or boost of any embodiment or combination disclosed herein. In some embodiments, the S protein is S2P. In some embodiments, the S protein is HexaPro.

The one or more doses of the DNA, mRNA, inactivated virus, adenoviral vector, or protein-based vaccine which is authorized for use to limit SARS-CoV2 infection may be administered to a subject treated in accordance with the methods described herein about 1-2 weeks, about 2-3 weeks, about 3-4 weeks, about 4-5 weeks, about 5-6 weeks, about 6-7 weeks, about 7-8 weeks, about 8-9 weeks, about 9-10 weeks, about 10-11 weeks, about 11-12 weeks, about 3-4 months, about 4-5 months, about 5-6 months, about 6-7 months, about 7-8 months, about 8-9 months about 9-10 months, about 10-11 months, about 11-12 months, about 12-15 months, about 15-18 months, about 18-21 months, about 21-24 months, about 2-3 years, about 3-4 years, or about 4-5 years before the administration of an immunogenic composition provided herein.

In some embodiments, the subject is a vaccination-naïve subject. In some embodiments, the subject is naïve to vaccinations to limit infection with a coronavirus. In some embodiments, the subject is naïve to vaccinations to limit infection with SARS-CoV2.

In some embodiments, the vaccine is a vaccination against an original strain of SARS-CoV2. In some embodiments, the vaccine is a vaccination against a variant strain of SARS-CoV2.

As used herein, the term “authorized for use” in the context of a vaccine means a vaccine has been approved for use in humans by a regulatory authority (e.g., the U.S. Food and Drug Administration, the European Medicines Agency, the Chinese National Medical Products Administration, the United Kingdom Medicines and Healthcare products Regulatory Agency, the Japanese Pharmaceutical Food and Medical Devices Agency, or the Russian Ministry of Health). Authorized for use can include emergency use authorization.

In some embodiments, a subject treated in accordance with the methods described herein has previously been infected with SARS-CoV-2. SARS-CoV-2 infection may be diagnosed using any PCR-based test or antigen-based test known in the art. In some embodiments, the subject has antibodies against SARS-CoV2 (e.g., an original strain or a variant strain) prior to the administering step. Anti-SARS-CoV2 antibodies may be detected using any serological test known in the art, including, for example, a test for IgM/IgG to the nucleocapsid protein, or a test for neutralizing antibodies against SARS-CoV2.

In some embodiments, a subject treated in accordance with the methods described herein has not previously been infected with SARS-CoV2. In some embodiments, the subject does not have antibodies against SARS-CoV2 prior to the administering step.

As used herein, “treat” or “treating” includes, but is not limited to accomplishing one or more of the following: (a) reducing SARS-CoV-2 titer in the subject; (b) limiting any increase of SARS-CoV-2 titer in the subject; (c) reducing the severity of SARS-CoV-2 symptoms; (d) limiting or preventing development of SARS-CoV-2 symptoms after infection; (e) inhibiting worsening of SARS-CoV-2 symptoms; (f) limiting or preventing recurrence of SARS-CoV-2 symptoms in subjects that were previously symptomatic for SARS-CoV-2 infection; and/or (e) survival.

As used herein, the term “full course” refers the one or more administrations (e.g., injections) of a vaccine or combination of vaccines as considered in the art to provide the desired level of protection against disease, such as a course of administration approved by a regulatory agency. For viral vectored vaccines, a full course may be a single administration. For nucleic acid-based vaccines (e.g. mRNA-based vaccines), a full course is generally two administrations spaced apart by about one month. Those skilled in the art are capable of recognizing a full course of vaccination. A full course of vaccination may include two, three, four, or more administrations. The compositions and method described herein may be employed in subjects who have received one, two, three, four, or more administrations of a prior vaccine or vaccines. In some examples, according to the Center for Disease Control, a subject is up to date COVID-19 vaccines when they have received all doses in the primary series and all boosters recommended, when eligible (fully vaccinated subject).

A method of treatment described herein may further comprise the administration of a second vaccination to the subject. In some embodiment, the second vaccination is administered concurrently with the SARS-CoV-2 vaccination.

In another aspect, provided herein is a method of vaccinating a subject, comprising administering to the subject (i) a pharmaceutical composition comprising an effective amount of a SARS-CoV-2 vaccine (for example, the protein complex comprising a first component comprising a receptor-binding domain of a coronavirus S protein and trimerization domain, and a second component comprising a pentamerization domain).

In some embodiments, the subject has received at least one dose of a vaccination for SARS-CoV-2 no less than 1 month, 2 months, 3 months, 4 months, 5 months, 6 months, 7 months, 8 months, 9 months, 10 months, 11 months, 12 months, 15 months, 18 months, 21 months, or 24 months before the administration of the combination of the SARS-CoV-2 vaccine and the second vaccine. In some embodiments, the subject has received at least one does of a vaccine comprising the receptor binding domain of a coronavirus S protein or a polynucleotide encoding the receptor binding domain of a coronavirus S protein, or a vaccine comprising a coronavirus S protein. The S protein may be, for example, S2P or HexaPro. The vaccine may be any approved vaccine for an original or a variant strain of SARS-CoV-2. The vaccine may be an mRNA-based vaccine, an adenoviral vector-based vaccine, a protein-based vaccine, or an inactivated virus vaccine. The subject may have received at least one dose, at least two doses, or at least three doses of the SARS-CoV-2 vaccine. In some embodiments, the subject has completed a full course of vaccination for an original or a variant SARS-CoV-2 strain. In some embodiments, the subject has been previously infected with SARS-CoV-2.

In further embodiments, the protein complexes and pharmaceutical compositions of the disclosure may be indicated for use in one or more of the following:

Primary immunization of SARS-CoV-2 naïve individuals.

Booster immunization to prevent COVID-19 caused by SARS-CoV-2 in previously SARS-CoV-2 vaccinated subjects.

Booster immunization to prevent COVID-19 caused by SARS-CoV-2 in previously SARS-CoV-2 vaccinated children (including individuals who have previously received booster doses).

Booster immunization to prevent COVID-19 caused by SARS-CoV-2 in previously SARS-CoV-2 vaccinated adolescents (including individuals who have previously received booster doses).

Booster immunization to prevent COVID-19 caused by SARS-CoV-2 in previously SARS-CoV-2 vaccinated adults 18+ years of age (including individuals who have previously received booster doses).

Booster immunization to prevent COVID-19 caused by SARS-CoV-2 in previously SARS-CoV-2 vaccinated children (including individuals who have previously received booster doses).

Booster vaccination in previously SARS-CoV-2 infected children.

Booster vaccination in previously SARS-CoV-2 infected adolescents.

Booster vaccination in previously SARS-CoV-2 infected adults 18+ years of age.

### Kits

The disclosure further provides kits, which may be used to prepare the virus-like particles and compositions of the disclosure. In some embodiments, a kit provided herein comprises a first component and a second component as disclosed herein, and instructions for use in a method of the disclosure. In some embodiment, a kit comprises one or more unit doses as disclosed herein, and instructions for use in a method of the disclosure. In some embodiments, the kit comprises a vial comprising a single dose of a pharmaceutical composition provided herein. In some embodiments, a kit comprises a vial comprising multiple doses provided herein. In some embodiments, a kit further comprises instructions for use of the pharmaceutical composition. In some embodiments, a kit further comprises a diluent for preparing dilutions of the pharmaceutical composition prior to administration. In some embodiments, the pharmaceutical composition comprises an adjuvant. In other embodiments, the kit comprises the composition comprising the protein complex and, separately, a composition comprising an adjuvant, such that the two compositions may be mixed prior to administration, or alternatively coadministered.

### Examples

### Example 1: Clinical Study

IVX-411 is a SARS-CoV-2 virus-like particle (VLP) vaccine targeting the original strain and incorporating the ACE2 receptor binding domain (RBD) from the SARS-CoV-2 S protein, a conserved antigen that induces neutralizing antibodies to several known epitopes, including those that prevent viral entry. The RBD protein is genetically fused to Component A and manufactured in mammalian cells. Component A-RBD is then combined with the same Component B used for our other programs to make the fully assembled VLPs, each of which incorporate 60 copies of the monomeric RBD antigen. The assembled protein complex is illustrated in FIG. 1. IVX-411 may be used in the clinic in both aqueous (non-adjuvanted) and adjuvanted formulations.

IVX-411 was tested in mice, rats, and nonhuman primates. Intramuscular injection of these VLPs induced strong neutralizing antibody responses, with titers observable after a single priming dose and significantly increased titers observable after a boosting dose. The immunogenicity generated in mice after vaccination with closely related precursor molecules, and formulated with an oil-in-water adjuvant has been shown to be durable, with neutralizing antibody titers remaining as high 20-24 weeks following the boosting dose as they were two weeks post-boost. In addition preclinical nonhuman primate data on a closely related precursor candidate assessed with several different adjuvant formulations have shown induction of robust neutralizing antibody titers well in excess of titers seen in human convalescent sera, and protection from viral challenge.

A GLP toxicology repeat intramuscular dose study was completed in rats. The study evaluated both injection site and systemic reactions to IVX-411, including non-adjuvanted and adjuvanted formulations. No test article-related effects were seen following administration of IVX-411 on mortality, clinical observations, ophthalmic observations, body weights, food consumption, or body temperature. No observed effects were considered adverse, and all observed effects were either partially or fully reversed 4 weeks following the last administration.

### Clinical Trials

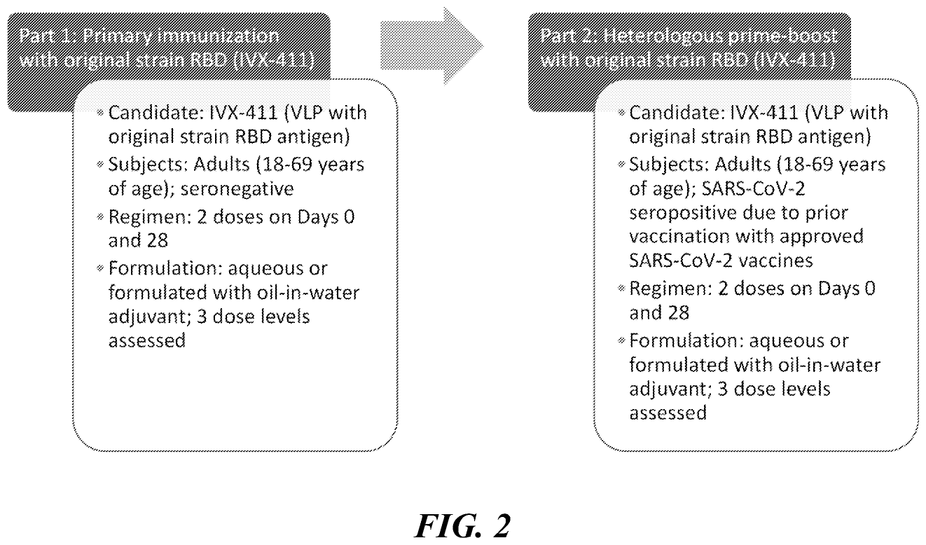

A Phase 1/2 trial is designed to evaluate the safety and immunogenicity of IVX-411 in primary and booster vaccinations. The clinical trial design is summarized in FIG. 2. There are two parts to the trial: Part 1 was a Phase 1 assessment of primary vaccination with IVX-411 in adults 18-69 years of age not previously exposed to SARS-CoV-2 (seronegative), and Part 2 was a Phase 2 assessment of IVX-411 booster vaccination in adults previously exposed through SARS-CoV-2 vaccination (seropositive). IVX-411 was administered as two doses, either unadjuvanted or formulated with an oil-in-water adjuvant, administered 28-days apart.

### Phase 1/2 Trial Design:

The Phase 1/2 trial was a randomized, placebo-controlled observer-blind dose-escalation study for safety and immunogenicity of two intramuscular (IM) doses of IVX-411. In Parts 1 and 2, six formulations of IVX-411 were tested including three dose levels each to be tested with and without Seqirus's proprietary adjuvant MF59®.

The candidate vaccine IVX-411 incorporates the angiotensin-converting enzyme 2 (ACE2) RBD from the SARS-CoV-2 spike (S) glycoprotein, a domain shown to be responsible for the majority (˜90%) of the nAbs against the virus found in human convalescent sera. There are two components that are assembled to form the VLP DS. The antigenic component (CompA-RBD-01) a structural component (CompB-01), when combined, self-assemble into an icosahedral VLP drug substance (DS). The IVX-411 DS is a VLP made of 20 copies of the CompA-RBD-01 DSI (displaying 60 copies of the RBD as 20 sets of 3 RBD antigens) and 12 copies of CompB-01 DSI.

The selected adjuvant, MF59® (MF59C.1®; Seqirus, Inc), is an oil-in-water emulsion with a squalene internal oil phase and a citric acid-sodium citrate buffer external aqueous phase.

Two drug products (DPs) were used for the phase 1/2 IVX-411-01 clinical study: IVX-411a (aqueous formulated DP) and IVX-411d (IVX-411a mixed 1:1 [V/V] with MF59® at the clinical site).

IVX-411a is an aqueous buffer formulation of IVX-411 DS filled in single use vials for IM use. IVX-411a DP is supplied in single-use 2.0 mL vials at a concentration of 500 μg/mL at a 0.5 mL fill volume. IVX-411d is a single-dose liquid formulation of IVX-411a that has been mixed with the MF59® adjuvant. MF59® is an oil-in-water emulsion with a squalene internal oil phase and a citric acid-sodium citrate buffer external aqueous phase.

Six IVX-411 formulations (Table 1) with and without MF59® were tested as follows:

TABLE 1

| Study Arm | VLP (μg) | MF59 (mg squalene) |
|---|---|---|
| A | 5 | 0 |
| B | 5 | 9.75 |
| C | 25 | 0 |
| D | 25 | 9.75 |
| E | 125 | 0 |
| F | 125 | 9.75 |
| G | 0 | 0 |

The study was conducted in two parts: Part 1 (first-in-human (FIH), Ph1) included healthy SARS-CoV-2-seronegative adults aged 18 to 69 years, inclusive. Part 2 (Booster, Ph2) included healthy adults aged 18 to 69 years inclusive, who were SARS-CoV-2-seropositive through prior vaccination with licensed SARS-CoV-2 vaccines. Both parts evaluated the safety and immunogenicity of two doses of IVX-411 vaccine administered 28 days apart, with or without MF59 adjuvant, in comparison with two doses of placebo.

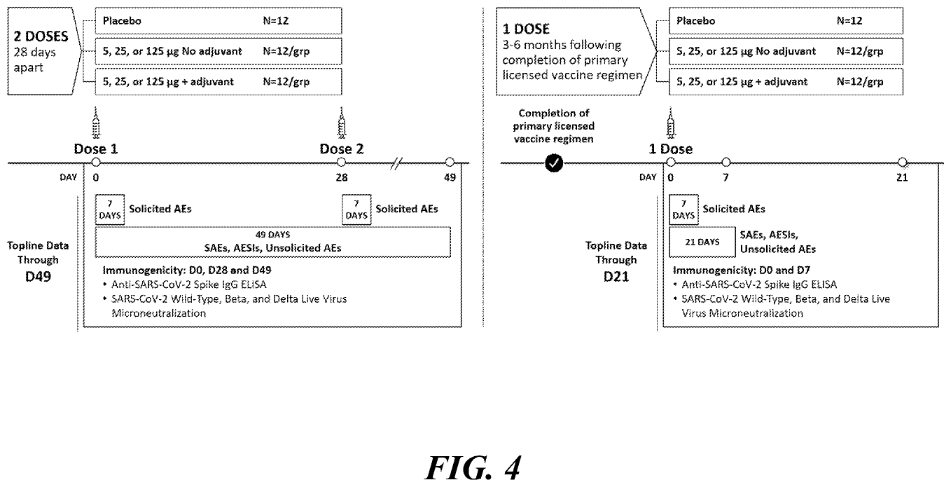

Part 1 of the Phase 1/2 (Ph1/2) study was a randomized, placebo-controlled observer-blind dose-escalation study for safety and immunogenicity of two intramuscular (IM) doses of IVX-411 administered 28-days apart (Day 0 and Day 28). The clinical trial design is summarized in FIG. 4.

Subjects in all study arms underwent blood sampling for serological immunogenicity testing and peripheral blood mononuclear cell isolation for evaluation. Safety and Efficacy were evaluated by: adverse events; SARS-CoV-2 neutralizing antibody (NAb) titers measured using a live-virus assay, and spike protein (S) specific and RBD-specific IgG antibody titers measured by multiplex assay. Efficacy will be further evaluated by: SARS-CoV-2 NAb titers by pseudovirion NAb assay; S-specific and RBD-specific IgG titers by enzyme-linked immunoassay (ELISA); and the ratios of fold-increase in RBD-specific IgG (multiplex assay) titers over fold-increase in SARS-CoV-2 NAb (live-virus assay) titers. Immunogenicity assays used are listed in Table 2.

TABLE 2

| Assay | Endpoint | Comment |
|---|---|---|
| SARS-CoV-2 live virus | Primary | Neutralization antibody titers that block SARS- |
| | | CoV-2 entry into cells |
| S-specific IgG multiplex | Primary | Evaluate the different S-specific isotypes |
| RBD-specific multiplex | Primary | Evaluate the different RBD-specific isotypes |
| S-specific IgG ELISA | Secondary | Total IgG response for analysis of quality metric |
| | | as defined by relative rise in ‘binding to |
| | | neutralizing’ ratio |
| RBD-specific ELISA | Secondary | Total IgG response for analysis of quality metric |
| | | as defined by relative rise in ‘binding to |
| | | neutralizing’ ratio |
| SARS-CoV-2 (variants) | Exploratory | Neutralization antibody titers that block SARS- |
| live virus | | CoV-2 entry into cells |

Part 2 was a Phase 2 assessment of booster vaccination with IVX-411 in 84 healthy adults who have been previously vaccinated with a licensed vaccine against SARS-CoV-2. The study determined whether an adjuvant was required in the formulation to enhance immune responses to IVX-411. The selected adjuvant, MF59®, was an oil-in-water emulsion. The clinical trial design is summarized in FIG. 4.

### Example 4: Immunogenicity and Safety of a Protein-Based Virus-Like Particle (VLP) SARS-CoV-2 Vaccine in Adults: A Phase 1/2 Study

This Example describes the results of the Phase 1/2 Study described in Example 2.

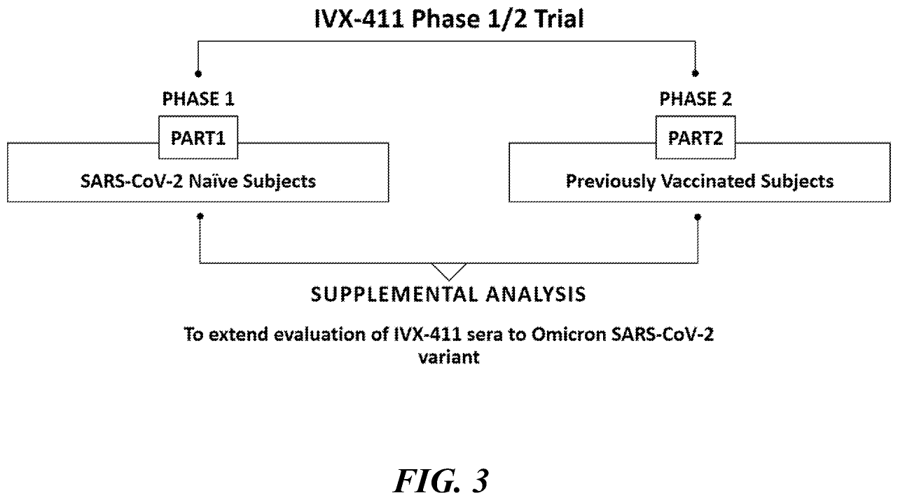

Background: COVID-19 continues to cause substantial morbidity and mortality globally. It is likely that booster vaccinations will be needed in future years to protect older adults and those with chronic medical conditions. We present interim topline results of a phase 1/2 study of IVX-411 [ACTRN12621000738820.; ACTRN12621000882820], an investigational VLP protein subunit SARS-CoV-2 vaccine, in adults aged 18-69 years (FIG. 3).

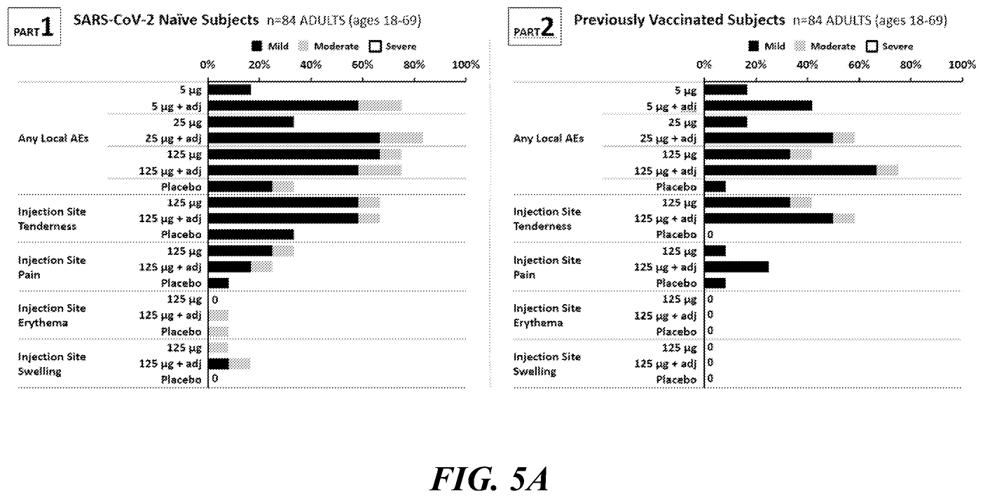

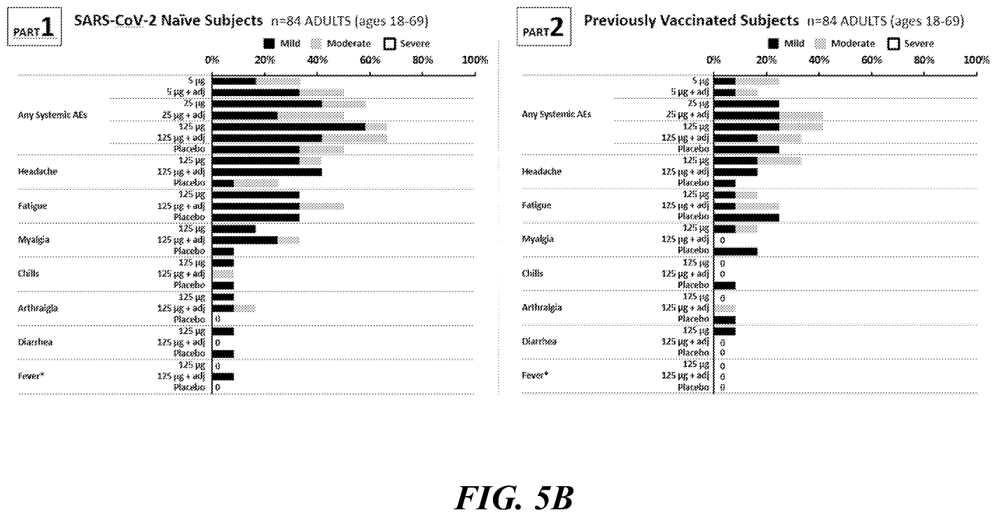

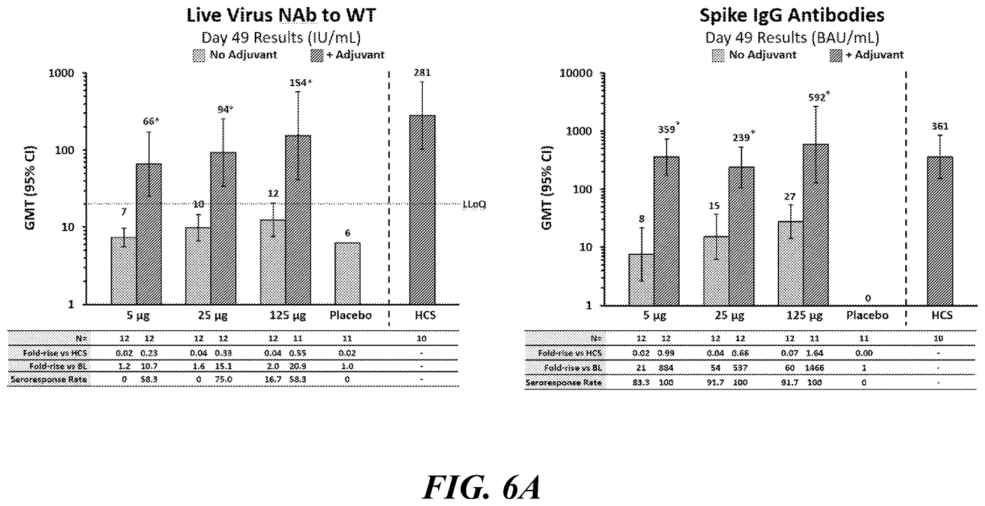

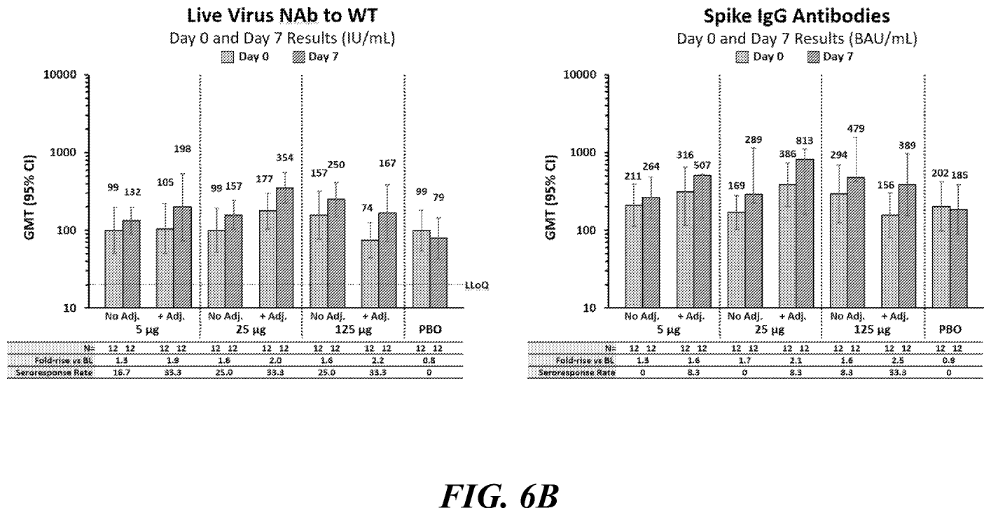

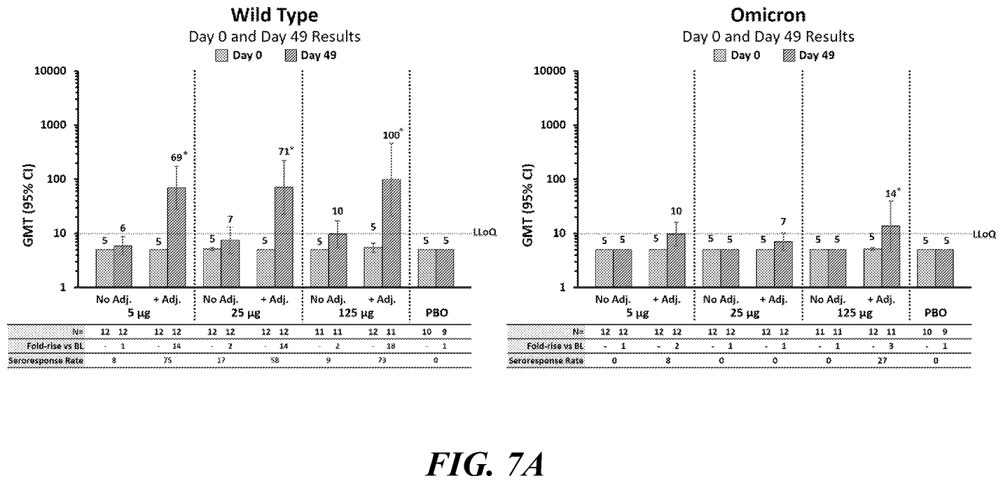

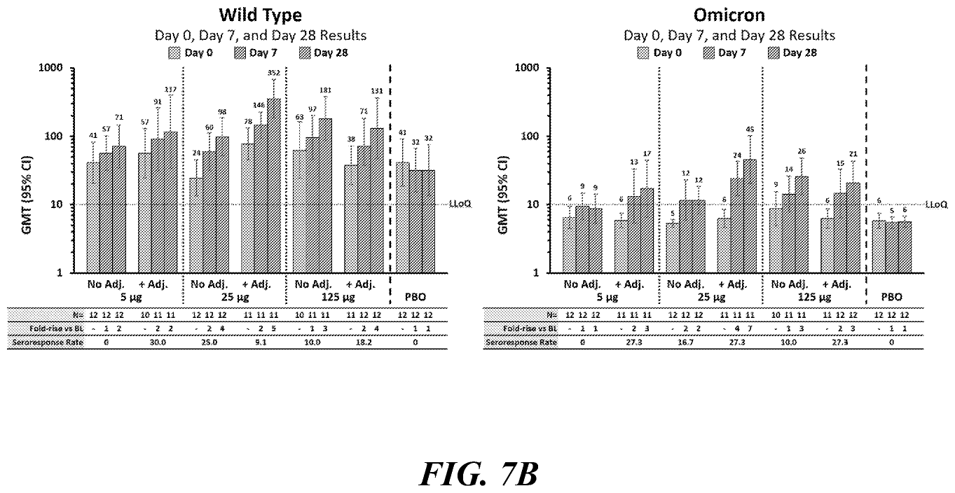

Methods: In Part 1, 84 SARS-CoV-2-naïve adults were randomized to receive two doses on Days 0 and 28 of either IVX-411 (5, 25, or 125 μg)±adjuvant, or placebo (FIG. 5, left panel). In Part 2, 84 subjects received a single dose of either IVX-411±adjuvant or placebo 3-6 months after completion of a primary licensed vaccine regimen (FIG. 5, right panel). Solicited adverse events (AEs) were collected for 7 days after each dose, with immunogenicity assessed on Days 0, 28, and 49 [(Part 1) and on Days 0, 7 and 28 (Part 2). Primary outcomes in both parts were solicited and unsolicited AEs, neutralizing antibody titers, and spike protein-specific IgG antibody titers.

Results: Demographics were similar in the IVX-411 groups versus placebo. In Part 1 and Part 2, local reactogenicity was mild-to-moderate, with higher rates of AEs with increasing doses and addition of adjuvant (FIG. 5A). Rates of systemic AEs were similar to placebo across groups (FIG. 5B). No vaccine-related severe or serious AEs were noted. IVX-411 was immunogenic in both primary and booster vaccination: in SARS-CoV-2-naïve subjects, a limited dose effect was seen, with significantly higher antibody titers in the groups receiving adjuvanted IVX-411 vaccine (p<0.01; FIG. 6A). The magnitude of antibody responses was similar to, or below, Human Convalescent Sera levels. In previously vaccinated subjects, IVX-411 boosted baseline antibody titers, with no conclusive dose or adjuvant effect (FIG. 6B). Immunogenicity was observed across all variants of concern (beta, delta, and omicron) in both parts, with up to 7-fold rises from baseline (FIGS. 13A and 13B).

Conclusion: The study met all primary safety and immunogenicity objectives, with acceptable tolerability profiles in primary and booster vaccination. A clear adjuvant effect was observed in SARS-CoV-2-naïve subjects. Clinical study met primary safety and immunogenicity objectives. Reactogenicity data was comparable to placebo for solicited and unsolicited events (Mild to moderate reactogenicity, none severe nor dose-limiting, no related serious adverse events or adverse events of special interest). Immunogenicity data showed immunogenicity in primary and booster vaccination (Neutralizing and IgG antibody titers exceed those of placebo recipients at Day 49 for WT, Clear evidence of adjuvant effect with more limited dose effect, with high rates of seroconversion, in primary regimen, Heterologous boosting after mRNA and adeno primary vaccination with up to 5-fold rise from baseline for WT, Immune responses seen across all variants of concern (beta, delta, omicron) in primary and booster context).

### ENUMERATED EMBODIMENTS

The disclosure further provides the following enumerated embodiments:

1. A pharmaceutical composition, comprising a protein complex comprising a first component comprising a receptor-binding domain of a coronavirus S protein attached to a first multimerization domain, and optionally a second component comprising a second multimerization domain; and optionally one or more pharmaceutically acceptable diluents or excipients.

2. The pharmaceutical composition of embodiment 1, wherein the pharmaceutical composition comprises an adjuvant.

3. The pharmaceutical composition of embodiment 2, wherein the adjuvant is a squalene-in-water emulsion.

4. The pharmaceutical composition of embodiment 3, wherein the adjuvant is MF59®.

5. The pharmaceutical composition of embodiment 2, wherein the adjuvant is an aluminum salt.

6. The pharmaceutical composition of embodiment 2, wherein the adjuvant is CPG-1018.

7. The pharmaceutical composition of embodiment 2, wherein the pharmaceutical composition comprises both an aluminum salt and CPG-1018.

8. The pharmaceutical composition of embodiment 1, wherein the pharmaceutical composition is free of or substantially free of any adjuvant.

9. The pharmaceutical composition of any one of embodiments 1 to 8, wherein the first multimerization domain is a trimerization domain and the second multimerization domain is a pentamerization domain.

10. The pharmaceutical composition of any one of embodiments 1 to 9, wherein the protein complex is an icosahedral protein complex.

11. The pharmaceutical compositions of any one of embodiments 1 to 10, wherein the first multimerization domain comprises an amino acid sequence which is at least 75%, 80%, 85%, 90%, 91%, 92%, 93%, 94%, 95%, 96%, 97%, 98%, 99%, or 100% identical to an amino acid sequence selected from the group consisting of SEQ ID NOS: 9-13 or 18.

12. The pharmaceutical compositions of any one of embodiments 1 to 11, wherein the second multimerization domain comprises an amino acid sequence which is at least 95%, at least 96%, at least 97%, at least 98%, at least 99%, or at least 100% identical to an amino acid sequence selected from the group consisting of SEQ ID NOS: 14-17, 20 or 27.

13. The pharmaceutical composition of any one of embodiments 1 to 12, wherein the first component comprises an amino acid sequence which is at least 75%, 80%, 85%, 90%, 91%, 92%, 93%, 94%, 95%, 96%, 97%, 98%, 99%, or 100% identical to any one of SEQ ID NOs: 1-6; and

wherein the second component comprises an amino acid sequence which is at least 75%, 80%, 85%, 90%, 91%, 92%, 93%, 94%, 95%, 96%, 97%, 98%, 99%, or 100% identical to SEQ ID NO: 14.

14. A unit dose of the pharmaceutical composition of any one of embodiments 1 to 13, wherein the unit dose comprises 2 μg, 5 μg, 10 μg, 15 μg, 25 μg, 50 μg, 100 μg, or 125 μg of the protein complex.

15. A method of vaccinating a subject at risk of infection with SARS-CoV-2, comprising administering to the subject a pharmaceutical composition comprising an effective amount of a protein complex comprising a first component comprising a receptor-binding domain of a coronavirus S protein attached to a first multimerization domain, and a second component comprising a second multimerization domain; and one or more pharmaceutically acceptable diluents or excipients.

16. A method of boosting an immune response to a prior vaccination for SARS-CoV-2, comprising administering to a subject previously vaccinated for SARS-CoV-2 a pharmaceutical composition comprising an effective amount of a protein complex comprising a first component comprising a receptor-binding domain of a coronavirus S protein attached to a first multimerization domain, and optionally a second component comprising a second multimerization domain.

17. The method of embodiments 16, therein the subject has been previously vaccinated with a full vaccination course of a primary vaccine.

18. A method of safely and effectively immunizing a subject for SARS-CoV-2, comprising administering to a subject previously vaccinated for SARS-CoV-2 a pharmaceutical composition comprising an effective amount of a protein complex comprising a first component comprising a receptor-binding domain of a coronavirus S protein attached to a first multimerization domain, and optionally a second component comprising a second multimerization domain.

19. The method of any one of embodiments 15 to 18, wherein the pharmaceutical composition comprises an adjuvant.

20. The method of embodiment 19, wherein the adjuvant is a squalene-in-water emulsion.

21. The method of embodiment 20, wherein the adjuvant is MF59®.

22. The method of embodiment 19, wherein the adjuvant is an aluminum salt.

23. The method of embodiment 19, wherein the adjuvant is CPG-1018.

24. The method of embodiment 19, wherein the pharmaceutical composition comprises both an aluminum salt and CPG-1018.

25. The method of embodiment 1, wherein the pharmaceutical composition is free of or substantially free of any adjuvant.

26. The method of any one of embodiments 15 to 25, wherein the first multimerization domain is a trimerization domain and the second multimerization domain is a pentamerization domain.

27. The method of any one of embodiments 15 to 26, wherein the protein complex is an icosahedral protein complex.

28. The method of any one of embodiments 15 to 27, wherein the first multimerization domain comprises an amino acid sequence which is at least 75%, 80%, 85%, 90%, 91%, 92%, 93%, 94%, 95%, 96%, 97%, 98%, 99%, or 100% identical to an amino acid sequence selected from the group consisting of SEQ ID NOS: 9-13 or 18.

29. The method of any one of embodiments 15 to 28, wherein the second multimerization domain comprises an amino acid sequence which is at least 95%, at least 96%, at least 97%, at least 98%, at least 99%, or at least 100% identical to an amino acid sequence selected from the group consisting of SEQ ID NOS: 14-17, 20 or 27.

30. The method of any one of embodiments 15 to 29, wherein the first component comprises an amino acid sequence which is at least 75%, 80%, 85%, 90%, 91%, 92%, 93%, 94%, 95%, 96%, 97%, 98%, 99%, or 100% identical to any one of SEQ ID NOs: 1-6; and

wherein the second component comprises an amino acid sequence which is at least 75%, 80%, 85%, 90%, 91%, 92%, 93%, 94%, 95%, 96%, 97%, 98%, 99%, or 100% identical to SEQ ID NO: 14.

31. The method of any one of embodiments 15 to 30, wherein the effective amount is 2 μg, 5 μg, 10 μg, 15 μg, 25 μg, 50 μg, 100 μg, or 125 μg of the protein complex.

32. The method of any one of embodiments 15 to 31, wherein the method comprises repeating the administering step.

33. The method of any one of embodiments 15 to 32, wherein the method comprises administering a booster vaccine.

34. The method of any one of embodiments 15 to 33, wherein the method comprises administering a prime vaccine.

35. The method of embodiment 34, wherein the prime vaccine is an mRNA-based vaccine, an adenoviral vector-based vaccine, a protein-based vaccine, or an inactivated virus vaccine.

36. The method of embodiment 34, wherein the prime vaccine is the protein complex.

37. The method of any one of embodiments 15 to 36, wherein the subject is a previously vaccinated subject.

38. The method of embodiment 37, wherein the subject has completed a full course of vaccination for an original strain of SARS-CoV-2.

39. The method of embodiment 38, wherein the subject has completed a partial course (e.g., has received one of two doses) of vaccination for an original strain of SARS-CoV-2.

40. The method of any one of embodiments 37 to 39, wherein the subject has received at least one dose of a vaccination for a variant strain of SARS-CoV-2.

41. The method any one of embodiments 37 to 40, wherein the subject has received at least one dose of a vaccine comprising the receptor binding domain of a coronavirus S protein or a polynucleotide encoding the receptor binding domain of a coronavirus S protein.

42. The method of any one of embodiments 37 to 40, wherein the subject has received at least one dose of a vaccine comprising a coronavirus S protein or a polynucleotide encoding a coronavirus S protein.

43. The method of embodiment 41 or 42, wherein the coronavirus S protein is S2P.

44. The method of embodiment 41 or 42, wherein the S protein is HexaPro.

45. The method of any one of embodiments 15 to 36, wherein the subject is a vaccination naïve subject.

46. The method of any one of embodiments 15 to 45, wherein the subject has previously been infected with SARS-CoV-2.

47. The method of any one of embodiments 15 to 46, wherein the subject has not previously been infected with SARS-CoV-2.

48. The method of any one of embodiments 15 to 47, wherein the subject does not have antibodies against SARS-CoV-2 prior to the administering step.

49. The method of any one of embodiments 15 to 47, wherein the subject has antibodies against SARS-CoV-2 prior to the administering step.

50. The method of any one of embodiments 15 to 49, wherein the method induces neutralizing antibody titers in the subject.

51. The method of any one of embodiments 15 to 50, wherein the method induces S protein-specific and IgG antibody titers in the subject.

52. The method of any one of embodiments 15 to 51, wherein the method prevents infection with SARS-CoV-2.

53. The method of embodiment 52, wherein the method prevents infection with an original strain of SARS-CoV-2.

54. The method of embodiment 52 or 53, wherein the method prevents infection with a variant strain of SARS-CoV-2.

55. The method of any one of embodiments 15 to 54, wherein the method reduces the severity of infection with coronavirus.

56. The method of embodiment 55, wherein the method reduces the severity of infection with an original strain of SARS-CoV-2.

57. The method of embodiment 55 or 56, wherein the method reduces the severity of infection with a variant strain of SARS-CoV-2.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

1. A pharmaceutical composition, comprising a protein complex comprising a first component comprising a receptor-binding domain of a coronavirus S protein attached to a first multimerization domain, and optionally a second component comprising a second multimerization domain; and one or more pharmaceutically acceptable diluents or excipients.

2. The pharmaceutical composition of claim 1, wherein the pharmaceutical composition comprises an adjuvant.

3. The pharmaceutical composition of claim 2, wherein the adjuvant is a squalene-in-water emulsion.

4. The pharmaceutical composition of claim 3, wherein the adjuvant is MF59®.

5. The pharmaceutical composition of claim 2, wherein the adjuvant is an aluminum salt.

6. The pharmaceutical composition of claim 2, wherein the adjuvant is CPG-1018.

7. The pharmaceutical composition of claim 2, wherein the pharmaceutical composition comprises both an aluminum salt and CPG-1018.

8. The pharmaceutical composition of claim 1, wherein the pharmaceutical composition is free of or substantially free of any adjuvant.

9. The pharmaceutical composition of any one of claims 1 to 8, wherein the first multimerization domain is a trimerization domain and the second multimerization domain is a pentamerization domain.

10. The pharmaceutical composition of any one of claims 1 to 9, wherein the protein complex is an icosahedral protein complex.

11. The pharmaceutical compositions of any one of claims 1 to 10, wherein the first multimerization domain comprises an amino acid sequence which is at least 75%, 80%, 85%, 90%, 91%, 92%, 93%, 94%, 95%, 96%, 97%, 98%, 99%, or 100% identical to an amino acid sequence selected from the group consisting of SEQ ID NOS: 9-13 or 18.

12. The pharmaceutical compositions of any one of claims 1 to 11, wherein the second multimerization domain comprises an amino acid sequence which is at least 95%, at least 96%, at least 97%, at least 98%, at least 99%, or at least 100% identical to an amino acid sequence selected from the group consisting of SEQ ID NOS: 14-17, 20 or 27.

13. The pharmaceutical composition of any one of claims 1 to 12, wherein the first component comprises an amino acid sequence which is at least 75%, 80%, 85%, 90%, 91%, 92%, 93%, 94%, 95%, 96%, 97%, 98%, 99%, or 100% identical to any one of SEQ ID NOs: 1-6; and
wherein the second component comprises an amino acid sequence which is at least 75%, 80%, 85%, 90%, 91%, 92%, 93%, 94%, 95%, 96%, 97%, 98%, 99%, or 100% identical to SEQ ID NO: 14.

14. A unit dose of the pharmaceutical composition of any one of claims 1 to 13, wherein the unit dose comprises 2 μg, 5 μg, 10 μg, 15 μg, 25 μg, 50 μg, 100 μg, or 125 μg of the protein complex.

15. A method of vaccinating a subject at risk of infection with SARS-CoV-2, comprising administering to the subject a pharmaceutical composition comprising an effective amount of a protein complex comprising a first component comprising a receptor-binding domain of a coronavirus S protein attached to a first multimerization domain, and a second component comprising a second multimerization domain; and one or more pharmaceutically acceptable diluents or excipients.

16. A method of boosting an immune response to a prior vaccination for SARS-CoV-2, comprising administering to a subject previously vaccinated for SARS-CoV-2 a pharmaceutical composition comprising an effective amount of a protein complex comprising a first component comprising a receptor-binding domain of a coronavirus S protein attached to a first multimerization domain, and optionally a second component comprising a second multimerization domain.

17. The method of claim 16, therein the subject has been previously vaccinated with a full vaccination course of a primary vaccine.

18. A method of safely and effectively immunizing a subject for SARS-CoV-2, comprising administering to a subject previously vaccinated for SARS-CoV-2 a pharmaceutical composition comprising an effective amount of a protein complex comprising a first component comprising a receptor-binding domain of a coronavirus S protein attached to a first multimerization domain, and optionally a second component comprising a second multimerization domain.

19. The method of any one of claims 15 to 18, wherein the pharmaceutical composition comprises an adjuvant.

20. The method of claim 19, wherein the adjuvant is a squalene-in-water emulsion.

21. The method of claim 20, wherein the adjuvant is MF59®.

22. The method of claim 19, wherein the adjuvant is an aluminum salt.

23. The method of claim 19, wherein the adjuvant is CPG-1018.

24. The method of claim 19, wherein the pharmaceutical composition comprises both an aluminum salt and CPG-1018.

25. The method of claim 1, wherein the pharmaceutical composition is free of or substantially free of any adjuvant.

26. The method of any one of claims 15 to 25, wherein the first multimerization domain is a trimerization domain and the second multimerization domain is a pentamerization domain.

27. The method of any one of claims 15 to 26, wherein the protein complex is an icosahedral protein complex.

28. The method of any one of claims 15 to 27, wherein the first multimerization domain comprises an amino acid sequence which is at least 75%, 80%, 85%, 90%, 91%, 92%, 93%, 94%, 95%, 96%, 97%, 98%, 99%, or 100% identical to an amino acid sequence selected from the group consisting of SEQ ID NOS: 9-13 or 18.

29. The method of any one of claims 15 to 28, wherein the second multimerization domain comprises an amino acid sequence which is at least 95%, at least 96%, at least 97%, at least 98%, at least 99%, or at least 100% identical to an amino acid sequence selected from the group consisting of SEQ ID NOS: 14-17, 20 or 27.

30. The method of any one of claims 15 to 29, wherein the first component comprises an amino acid sequence which is at least 75%, 80%, 85%, 90%, 91%, 92%, 93%, 94%, 95%, 96%, 97%, 98%, 99%, or 100% identical to any one of SEQ ID NOs: 1-6; and
wherein the second component comprises an amino acid sequence which is at least 75%, 80%, 85%, 90%, 91%, 92%, 93%, 94%, 95%, 96%, 97%, 98%, 99%, or 100% identical to SEQ ID NO: 14.

31. The method of any one of claims 15 to 30, wherein the effective amount is 2 μg, 5 μg, 10 μg, 15 μg, 25 μg, 50 μg, 100 μg, or 125 μg of the protein complex.

32. The method of any one of claims 15 to 31, wherein the method comprises repeating the administering step.

33. The method of any one of claims 15 to 32, wherein the method comprises administering a booster vaccine.

34. The method of any one of claims 15 to 33, wherein the method comprises administering a prime vaccine.

35. The method of claim 34, wherein the prime vaccine is an mRNA-based vaccine, an adenoviral vector-based vaccine, a protein-based vaccine, or an inactivated virus vaccine.

36. The method of claim 34, wherein the prime vaccine is the protein complex.

37. The method of any one of claims 15 to 36, wherein the subject is a previously vaccinated subject.

38. The method of claim 37, wherein the subject has completed a full course of vaccination for an original strain of SARS-CoV-2.

39. The method of claim 38, wherein the subject has completed a partial course (e.g., has received one of two doses) of vaccination for an original strain of SARS-CoV-2.

40. The method of any one of claims 37 to 39, wherein the subject has received at least one dose of a vaccination for a variant strain of SARS-CoV-2.

41. The method any one of claims 37 to 40, wherein the subject has received at least one dose of a vaccine comprising the receptor binding domain of a coronavirus S protein or a polynucleotide encoding the receptor binding domain of a coronavirus S protein.

42. The method of any one of claims 37 to 40, wherein the subject has received at least one dose of a vaccine comprising a coronavirus S protein or a polynucleotide encoding a coronavirus S protein.

43. The method of claim 41 or 42, wherein the coronavirus S protein is S2P.

44. The method of claim 41 or 42, wherein the S protein is HexaPro.

45. The method of any one of claims 15 to 36, wherein the subject is a vaccination naïve subject.

46. The method of any one of claims 15 to 45, wherein the subject has previously been infected with SARS-CoV-2.

47. The method of any one of claims 15 to 46, wherein the subject has not previously been infected with SARS-CoV-2.

48. The method of any one of claims 15 to 47, wherein the subject does not have antibodies against SARS-CoV-2 prior to the administering step.

49. The method of any one of claims 15 to 47, wherein the subject has antibodies against SARS-CoV-2 prior to the administering step.

50. The method of any one of claims 15 to 49, wherein the method induces neutralizing antibody titers in the subject.

51. The method of any one of claims 15 to 50, wherein the method induces S protein-specific and IgG antibody titers in the subject.

52. The method of any one of claims 15 to 51, wherein the method prevents infection with SARS-CoV-2.

53. The method of claim 52, wherein the method prevents infection with an original strain of SARS-CoV-2.

54. The method of claim 52 or 53, wherein the method prevents infection with a variant strain of SARS-CoV-2.

55. The method of any one of claims 15 to 54, wherein the method reduces the severity of infection with coronavirus.

56. The method of claim 55, wherein the method reduces the severity of infection with an original strain of SARS-CoV-2.

57. The method of claim 55 or 56, wherein the method reduces the severity of infection with a variant strain of SARS-CoV-2.


SEQUENCE LISTING (28 sequences)

SEQ ID NO: 1 - protein, 435 aa, Artificial Sequence
  receptor-binding domain of a coronavirus S protein
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 2 - protein, 435 aa, Artificial Sequence
  receptor-binding domain of a coronavirus S protein
RFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 3 - protein, 435 aa, Artificial Sequence
  receptor-binding domain of a coronavirus S protein
RFPNITNLCPFGEVFNATRFASVYAWNRKRISNCVADFSVLYNSASFSTFKCYGVSPTKLNDLCWTNIYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 4 - protein, 435 aa, Artificial Sequence
  receptor-binding domain of a coronavirus S protein
RFPNITNLCPFGEVFNATRFASVYAWNRKRISNCVADFSVLYNSASFSTFKCYGVSPTKLNDLCWTNIYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 5 - protein, 435 aa, Artificial Sequence
  receptor-binding domain of a coronavirus S protein
RFPNITNLCPFGEVFNATRFASVYAWNRKRISNCVADFSVLYNSASFSTFKCYGVSPTKLNDLCWTNIYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 6 - protein, 435 aa, Artificial Sequence; identical to SEQ ID NO: 4
  receptor-binding domain of a coronavirus S protein

SEQ ID NO: 7 - protein, 1016 aa, Artificial Sequence
  wild-type SARS-CoV-2 S protein, subunit 1
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAA

SEQ ID NO: 8 - protein, 12 aa, Artificial Sequence
  foldon
EKAAKAEEAARK

SEQ ID NO: 9 - protein, 202 aa, Artificial Sequence
  I53-50A
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 10 - protein, 202 aa, Artificial Sequence
  I53-50A.1
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCEWFKAGVLAVGVGDALVKGDPDEVREKAKKFVEKIRGCTE

SEQ ID NO: 11 - protein, 202 aa, Artificial Sequence
  I53-50A.1NegT2
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVEAMKGPFPNVKFVPTGGVDLDDVCEWFDAGVLAVGVGDALVEGDPDEVREDAKEFVEEIRGCTE

SEQ ID NO: 12 - protein, 202 aa, Artificial Sequence
  I53-50A.1PosT1
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGKALVKGKPDEVREKAKKFVKKIRGCTE

SEQ ID NO: 13 - protein, 202 aa, Artificial Sequence
  I53-50A genus
  MISC_FEATURE (123)..(123): Xaa is T or D
  MISC_FEATURE (136)..(136): Xaa is Q or E
  MISC_FEATURE (139)..(139): Xaa is K or E
  MISC_FEATURE (157)..(157): Xaa is N or D
  MISC_FEATURE (160)..(160): Xaa is N or D
  MISC_FEATURE (163)..(163): Xaa is E or K
  MISC_FEATURE (166)..(166): Xaa is D or K
  MISC_FEATURE (176)..(176): Xaa is S, D or K
  MISC_FEATURE (180)..(180): Xaa is K or E
  MISC_FEATURE (182)..(182): Xaa is T, D, or K
  MISC_FEATURE (189)..(189): Xaa is D or K
  MISC_FEATURE (192)..(192): Xaa is A, E,  or K
  MISC_FEATURE (195)..(195): Xaa is E or K
  MISC_FEATURE (196)..(196): Xaa is E or K
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHXILKLFPGEVVGPXFVXAMKGPFPNVKFVPTGGVXLDXVCXWFXAGVLAVGVGXALVXGXPDEVREXAKXFVXXIRGCTE

SEQ ID NO: 14 - protein, 156 aa, Artificial Sequence
  I53-50B
NQHSHKDYETVRIAVVRARWHAEIVDACVSAFEAAMADIGGDRFAVDVFDVPGAYEIPLHARTLAETGRYGAVLGTAFVVNGGIYRHEFVASAVIDGMMNVQLSTGVPVLSAVLTPHRYRDSDAHTLLFLALFAVKGMEAARACVEILAAREKIAA

SEQ ID NO: 15 - protein, 156 aa, Artificial Sequence
  I53-50B.1
NQHSHKDHETVRIAVVRARWHAEIVDACVSAFEAAMRDIGGDRFAVDVFDVPGAYEIPLHARTLAETGRYGAVLGTAFVVNGGIYRHEFVASAVIDGMMNVQLDTGVPVLSAVLTPHRYRDSDAHTLLFLALFAVKGMEAARACVEILAAREKIAA

SEQ ID NO: 16 - protein, 156 aa, Artificial Sequence
  I53-50B.1NegT2
NQHSHKDHETVRIAVVRARWHAEIVDACVSAFEAAMRDIGGDRFAVDVFDVPGAYEIPLHARTLAETGRYGAVLGTAFVVDGGIYDHEFVASAVIDGMMNVQLDTGVPVLSAVLTPHEYEDSDADTLLFLALFAVKGMEAARACVEILAAREKIAA

SEQ ID NO: 17 - protein, 156 aa, Artificial Sequence
  I53-50B.4PosT1
NQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 18 - protein, 156 aa, Artificial Sequence
  I53-50B  genus
  MISC_FEATURE (8)..(8): Xaa is Y or H
  MISC_FEATURE (37)..(37): Xaa is A or R
  MISC_FEATURE (81)..(81): Xaa is N or D
  MISC_FEATURE (86)..(86): Xaa is R or D
  MISC_FEATURE (96)..(96): Xaa is N or D
  MISC_FEATURE (104)..(104): Xaa is S, N, or D
  MISC_FEATURE (118)..(118): Xaa is R, E, or N
  MISC_FEATURE (120)..(120): Xaa is R, E, or D
  MISC_FEATURE (121)..(121): Xaa is K or D
  MISC_FEATURE (123)..(123): Xaa is K or D
  MISC_FEATURE (125)..(125): Xaa is H or D
NQHSHKDXETVRIAVVRARWHAEIVDACVSAFEAAMXDIGGDRFAVDVFDVPGAYEIPLHARTLAETGRYGAVLGTAFVVXGGIYXHEFVASAVIXGMMNVQLXTGVPVLSAVLTPHXYXXSXAXTLLFLALFAVKGMEAARACVEILAAREKIAA

SEQ ID NO: 19 - protein, 119 aa, Artificial Sequence
  I53_dn5B
EEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 20 - protein, 153 aa, Artificial Sequence
  I53_dn5A
KYDGSKLRIGILHARWNAEIILALVLGALKRLQEFGVKRENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 21 - protein, 204 aa, Artificial Sequence
  receptor-binding domain of a coronavirus S protein
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 22 - protein, 204 aa, Artificial Sequence
  receptor-binding domain of a coronavirus S protein
RFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKST

SEQ ID NO: 23 - protein, 204 aa, Artificial Sequence
  receptor-binding domain of a coronavirus S protein
RFPNITNLCPFGEVFNATRFASVYAWNRKRISNCVADFSVLYNSASFSTFKCYGVSPTKLNDLCWTNIYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKST

SEQ ID NO: 24 - protein, 204 aa, Artificial Sequence
  receptor-binding domain of a coronavirus S protein
RFPNITNLCPFGEVFNATRFASVYAWNRKRISNCVADFSVLYNSASFSTFKCYGVSPTKLNDLCWTNIYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKST

SEQ ID NO: 25 - protein, 28 aa, Artificial Sequence
  signal peptide
MGILPSPGMPALLSLVSLLSVLLMGCVA

SEQ ID NO: 26 - protein, 32 aa, Artificial Sequence
  signal peptide
MGILPSPGMPALLSLVSLLSVLLMGCVAETGT

SEQ ID NO: 27 - protein, 157 aa, Artificial Sequence
  I53-50B.4PosT1
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 28 - protein, 204 aa, Artificial Sequence
  I53-50A-delta cys
EELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELE